(19) United States
(12) Patent Application Publication
(10) Pub. No.: US 2002/0147169 A1
(43) Pub. Date: Oct. 10, 2002

(54) In vivo selection method for determining inhibitory RNA molecules

(76) Inventors: Kyri Mitrophanous, Oxford (GB); Narry Kim, Philadelphia, PA (US); Ekaterini Kotsopoulou, Boston, MA (US)

Correspondence Address: FROMMER LAWRENCE & HAUG, 745 FIFTH AVENUE- 10TH FL., NEW YORK, NY, 10151, US

(21) Appl. No.: 09/999,183

(22) Filed: Nov. 29, 2001

Related U.S. Application Data
(63) Continuation-in-part of application No. PCT/GB00/02136, filed on Jun. 2, 2000.

(30) Foreign Application Priority Data
Jun. 3, 1999 (GB) 9912965.2

(51) Int. Cl. A61K 48/00; C12N 15/00; A61K 31/70; A01N 43/04; C12N 15/09; C12N 15/63; C12N 15/70; C12N 15/74; C12N 15/85; C12N 15/87

(52) U.S. Cl. 514/44; 435/320.1; 435/455

(57) ABSTRACT

A selection system suitable for use in vivo is provided, the system comprising: I) a plurality of first nucleotide sequences encoding a gene product capable of binding to and effecting the cleavage, directly or indirectly, of a nucleotide sequence, or a transcription product thereof; wherein a region of the first nucleotide sequence required for binding to the nucleotide sequence is heterogeneous within the plurality of first nucleotide sequences; and ii) a second nucleotide sequence comprising: (a) a coding region encoding a detectable marker operably linked to sequences required for mRNA stability and/or translation; and (b) a third nucleotide sequence positioned between the coding region and at least one of the sequences required for mRNA stability and/or translation; wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof.

### RELATED APPLICATIONS

This application is a Continuation-in-Part Application of International Application PCT/GB00/02136 filed Jun. 2, 2000, published as WO 00/75370 A1 on Dec. 14, 2000, and claims priority from Great Britain Application 9912965.2, filed Jun. 3, 1999. Each of the foregoing applications, patents and publications and all documents cited or referenced therein (“application cited documents”) and all documents cited or referenced in this specification (“herein cited documents”) and all documents referenced or cited in herein cited documents and in application cited documents, including during the prosecution of any of the applications, patents and application cited documents, are hereby incorporated herein by reference.

### FIELD OF THE INVENTION

The present invention relates to an in vivo method for selecting optimal inhibitory RNA molecules.

### BACKGROUND TO THE INVENTION

There is considerable interest in the use of inhibitory RNA molecules to inhibit the transcription and/or translation of target nucleic acid sequences. An inhibitory RNA molecule may be capable of binding to and effecting the cleavage, directly or indirectly, of a nucleotide sequence, or its transcription product. Examples include ribozymes (which are capable of direct cleavage of the target sequence), external guide sequences (EGSs) and anti-sense or sense RNA (which are capable of causing cleavage by other factors).

Unfortunately the application of such technology has encountered difficulties due to the complex secondary structure of mRNA. It is currently impossible to predict the location of sites within mRNA that are open or accessible other than by trying to target every location.

One such approach used to search for these open areas is the in vitro ‘Oligonucleotide Arrays’ method described by Southern (1997) and Milner (1997). However, these methods involve the time consuming generation of unique arrays for each particular target mRNA. In addition the formation of heteroduplexes is carried out in non-physiological conditions. This is especially problematic when one considers that mRNA has a number of proteins associated at both the 5′ and 3′ ends.

It would therefore be advantagous to use an ‘in vivo’ selection method to discover open mRNA sites in their normal physiological context. In addition the method should be generally useful in that all RNA molecules could be scanned in this way without having to resort to particular selection methods each time.

### SUMMARY OF THE INVENTION

It is therefore an object of the present invention to provide an in vivo selection method for identifying inhibitory RNA molecules that target efficiently a given RNA sequence.

The present inventors have shown that it is possible to select inhibitory RNA molecules (such as ribozymes) in vivo using cells which express a target sequence operably linked to a detectable marker, such that the target sequence and detectable marker are expressed as a contiguous RNA molecule in the cells.

The target sequence/detectable marker mRNA will contain the normal 5′ cap and the 3′ poly A tail. If the 5′ cap or the 3′ poly A tail are removed (for example following cleavage of the target sequence by an inhibitory RNA molecule) the mRNA will become unstable and will be degraded. This will prevent translation of the detectable marker gene.

If, for example, the detectable marker is an enzyme capable of converting a prodrug into a cytotoxic compound, cleavage of the target sequence will render the cell insensitive to the prodrug.

Cells expressing the detectable marker/target gene may be used to screen a large number of inhibitory RNA molecules. In particular, the present inventors have used such cells to screen a library of ribozyme-expressing vectors with each ribozyme having a different specificity region. Cells which are resistant to the prodrug will contain an active ribozyme, the sequence of which can then be determined using techniques such as the polymerase chain reaction (PCR).

Accordingly the present invention provides a selection system suitable for use in vivo, the system comprising:

(i) a plurality of first nucleotide sequences encoding a gene product capable of binding to and effecting the cleavage, directly or indirectly, of a nucleotide sequence, or a transcription product thereof,

wherein a region of the first nucleotide sequence required for binding to the nucleotide sequence is heterogeneous within the plurality of first nucleotide sequences; and

(ii) a second nucleotide sequence comprising

(a) a coding region encoding a detectable marker operably linked to sequences

required for mRNA stability and/or translation: and

(b) a third nucleotide sequence positioned between the coding region and at least one of the sequences required for mRNA stability and/or translation;

wherein (a) and (b) are operably linked to a regulatory sequence capable of directing expression of (a) and (b) as a contiguous RNA molecule in a host cell.

The present invention also provides a vector system comprising:

(i) a plurality of vectors, each vector independently comprising

a first nucleotide sequence encoding a gene product capable of binding to and effecting the cleavage, directly or indirectly, of a nucleotide sequence, or a transcription product thereof,

wherein a region of the first nucleotide sequence required for binding to the nucleotide sequence is heterogeneous within the plurality of vectors; and

(ii) a second nucleotide sequence comprising

(a) a coding region encoding a detectable marker operably linked to sequences

required for mRNA stability and/or translation; and

(b) a third nucleotide sequence positioned between the coding region and at least one of the sequences required for mRNA stability and/or translation;

wherein (a) and (b) are operably linked to a regulatory sequence capable of directing expression of (a) and (b) as a contiguous RNA molecule in a host cell, wherein the second nucleotide sequence is present in the plurality of vectors or as part of a separate vector.

Preferably the gene product is selected from a ribozyme, an anti-sense ribonucleic acid and an external guide sequence.

Preferably, the vector is a viral vector, more preferably a retroviral vector.

Preferably the detectable marker is a selectable marker, more preferably an enzyme capable of converting a prodrug into a cytotoxic compound. In a particularly preferred embodiment the enzyme is thymidine kinase and the prodrug is gancyclovir.

The present invention further provides a plurality of viral particles, each viral particle comprising a first nucleotide sequence and/or a second nucleotide sequence as defined above.

The present invention also provides a method for producing plurality of viral particles as defined above which method comprises introducing into a producer cell (i) a plurality of first nucleotide sequences as defined above and (ii) a second nucleotide sequence as defined above. A plurality of viral particles produced by this method is also provided.

In a further aspect, the present invention provides a method for selecting from a plurality of first nucleotide sequences as defined above, a first nucleotide sequence encoding a gene product capable of binding to and effecting the cleavage, directly or indirectly, of a third nucleotide sequence, or a transcription product thereof; which method comprises:

(i) introducing one or more first nucleotide sequences into a host cell comprising a second nucleotide sequence as defined above; and

(ii) selecting the host cell if the detectable marker is not expressed in an active form in the host cell; and optionally,

(iii) isolating the first nucleotide sequence and determining its nucleotide sequence.

Preferably, in step (ii), the detectable marker is a selectable marker and the host cell is contacted with a compound such as a prodrug which is cytotoxic in the presence of the selectable marker. More preferably, the selectable marker is thymidine kinase and the prodrug is gancyclovir.

In a preferred embodiment, each first nucleotide sequence in the plurality of first nucleotide sequences is located downstream of a fourth nucleotide sequence encoding an inactive variant of the detectable marker such that any first nucleotide sequence(s) encoding a gene product capable of binding to and effecting the cleavage of a coding region encoding the detectable marker is removed.

In an especially preferred aspect of the invention, the plurality of first nucleotide sequences are present in a plurality of viral vectors, said viral vectors comprising a fourth nucleotide sequence encoding an inactive variant of the detectable marker, and as a preliminary step, the plurality of vectors are introduced into one or more producer cells and any resulting infectious viral particles used in step (i) to introduce the first nucleotides sequences into producer cells comprising a second nucleotide sequence.

The present invention provides in another aspect, a first nucleotide sequence obtained by the above method of the invention. Such a first nucleotide sequence may be used in therapy.

In a further aspect, the present invention provides a method for identifying an open site on a mRNA molecule, using a selection system according to the first aspect of the invention.

### DETAILED DESCRIPTION OF THE INVENTION

A. Components of Selection System

(i) Plurality of First Nucleotide Sequences Encoding Inhibitory RNA Molecules

The first component of the selection system of the present invention is a plurality of nucleotide sequences which encode inhibitory RNA molecules. An inhibitory RNA molecule is defined as a ribonucleic acid which is capable of inhibiting transcription and/or translation of a nucleic acid sequence by binding to the sequence. Generally, an inhibitory RNA molecule is capable of binding to and effecting the cleavage, directly or indirectly, of a nucleotide sequence, or its transcription product. Examples include ribozymes (cleave directly), external guide sequences (EGSs) and anti-sense or sense RNA (cause cleavage by other factors).

The plurality of first nucleotide sequences encoding inhibitory RNA molecules will be substantially heterogeneous to create a diversity of sequences for the selection process. Thus, at least the part of the inhibitory molecule required for binding to a target nucleotide sequence will vary. Preferably, sequences required for catalytic activity, structural integrity and interactions with cellular components that degrade the target nucleotide sequence are not varied. However it may be convenient to produce the library by mutagenesis techniques which make randomly alterations throughout the inhibitory RNA molecule sequence. In the case of antisense and sense RNA constructs, this is in any case generally desirable. With regard to ribozymes and EGS sequences, it is preferred to generate the library by the use of randomly synthesised oligonucleotides which are then ligated into ribozyme or EGS constructs in the appropriate position. This results in a library of ribozymes/EGSs constructs where the region required for specificity varies whilst the other functional domains typically are constant.

By way of an example, a random ribozyme library can be generated by constructing oligonucleotides that contain the conserved enzymatic helix of the hammerhead ribozyme flanked with random nucleotides. The flanking sequences provide the recognition regions for the ribozyme. The length of each of the flanking sequences can vary although 8 nucleotides is a suitable length. This gives a complexity of approximately 16⁴ (65536) different molecules.

Suitable first nucleotide sequences for use according to the present invention encode gene products that result in the cleavage and/or enzymatic degradation of a target nucleotide sequence, which will generally be a ribonucleotide. As particular examples, ribozymes, external guide sequences and antisense sequences may be mentioned.

Ribozymes are RNA enzymes which cleave RNA at specific sites. Ribozymes can be engineered so as to be specific for any chosen sequence containing a ribozyme cleavage site. Thus, ribozymes can be engineered which have chosen recognition sites in transcribed viral sequences. By way of an example, ribozymes encoded by the first nucleotide sequence recognise and cleave essential elements of viral genomes required for the production of viral particles, such as packaging components and the mRNA genome. Thus, for retroviral genomes, such essential elements include the gag, pol and env gene products, and the viral mRNA genome. A suitable ribozyme capable of recognising at least one of the gag, pol and env gene sequences, or more typically, the RNA sequences transcribed from these genes, is able to bind to and cleave such a sequence. This will reduce or prevent production of the gal, pol or env protein as appropriate as well as the mRNA genome and thus reduce or prevent the production of retroviral particles. Ribozymes come in several forms, including hammerhead hairpin and hepatitis delta antigenomic ribozymes. Preferred for use herein are hammerhead ribozymes, in part because of their relatively small size, because the sequence requirements for their target cleavage site are minimal and because they have been well characterised. The ribozymes most commonly used in research at present are hammerhead and hairpin ribozymes.

Each individual ribozyme has a motif which recognises and binds to a recognition site in the target RNA. This motif takes the form of one or more “binding arms”, generally two binding arms. The binding arms in hammerhead ribozymes are the flanking sequences Helix I and Helix III, which flank Helix III. These can be of variable length, usually between 6 to 10 nucleotides each, but can be shorter or longer. The length of the flanking sequences can affect the rate of cleavage. For example, it has been found that reducing the total number of nucleotides in the flanking sequences from 20 to 12 can increase the turnover rate of the ribozyme cleaving a HIV sequence, by 10-fold. A catalytic motif in the ribozyme Helix II in hammerhead ribozymes cleaves the target RNA at a site which is referred to as the cleavage site. Whether or not a ribozyme will cleave any given RNA is determined by the presence or absence of a recognition site for the ribozyme containing an appropriate cleavage site.

Each type of ribozyme recognises its own cleavage site. The hammerhead ribozyme cleavage site has the nucleotide base triplet GUX directly upstream where G is guanine, U is uracil and X is any nucleotide base. Hairpin ribozymes have a cleavage site of BCUGNYR, where B is any nucleotide base other than adenine, N is any nucleotide, Y is cytosine or thymine and R is guanine or adenine. Cleavage by hairpin ribozymes takes places between the G and the N in the cleavage site.

Antisense technology is well known in the art. One mechanism by which antisense sequences are believed to function is the recruitment of the cellular protein RNAseH to the target sequence/antisense construct heteroduplex which results in cleavage and degradation of the heteroduplex. Thus the antisense construct, by contrast to ribozymes, can be said to lead indirectly to cleavage/degradation of the target sequence. Thus according to the present invention, a first nucleotide sequence may encode an antisense RNA that binds to either a gene encoding an essential/packaging component or the RNA transcribed from said gene such that expression of the gene Is inhibited, for example as a result of RNAseH degradation of a resulting heteroduplex. It is not necessary for the antisense construct to encode the entire complementary sequence of the gene encoding an essential/packaging component—a portion may suffice. The skilled person will easily be able to determine how to design a suitable antisense construct.

External guide sequences (EGSs) are RNA sequences that bind to a complementary target sequence to form a loop in the target RNA sequence, the overall structure being a substrate for RNaseP-mediated cleavage of the target RNA sequence. The structure that forms when the EGS anneals to the target RNA is very similar to that found in a tRNA precursor. The the natural activity of RNaseP can be directed to cleave a target RNA by designing a suitable EGS. The general rules for EGS design are as follows, with reference to the generic EGSs shown in FIG. 13:

### Rules for EGS Design in Mammalian Cells (see FIG. 13)

Target sequence—All tRNA precursor molecules have a G immediately 3′ of the RNaseP cleavage site (i.e. the G forms a base pair with the C at the top of the acceptor stem prior to the ACCA sequence). In addition a U is found 8 nucleotides downstream in all tRNAs. (i.e. G at position 1, U at position 8). A pyrimidine may be preferred 5′ of the cut site. No other specific target sequences are generally required.

EGS sequence—A 7 nucleotide ‘acceptor stem’ analogue is optimal (5′ hybridising arm). A 4 nucleotide ‘D-stem’ analogue is preferred (3′ hybridising arm). Variation in this length may alter the reaction kinetics. This will be specific to each target site. A consensus ‘T-stem and loop’ analogue is essential. Minimal 5′ and 3 ′ non-pairing sequences are preferred to reduce the potential for undesired folding of the EGS RNA.

Deletion of the ‘anti-codon stem and loop’ analogue may be beneficial. Deletion of the variable loop can also be tolerated in vitro but an optimal replacement loop for the deletion of both has not been defined in vivo. Further guidance may be obtained by reference to, for example, Werner et al. (1997); Werner et al. (1998); Ma et al. (1998) and Kawa et al. (1998).

The nucleotide sequences will typically be part of a vector construct which will thus comprise a first nucleotide sequence encoding an inhibitory RNA molecule operably linked to a regulatory control sequence which permits expression of the inhibitory RNA molecule in the host cell. Further details of vector constructs, including viral vector constructs, are given below in section B.

ii. Second Nucleotide Sequence Comprising Reporter Constructs

The second nucleotide sequence in the system essential functions as a reporter construct. Thus the second nucleotide sequence comprises a coding region encoding a detectable marker. A detectable marker is any gene product which expression can be detected. Examples include lacZ green fluorescent protein and selectable markers. Preferably the detectable marker is sortable using fluorescence activated cell sorting (FACS).

Selectable markers are typically gene products whose expression, or lack of, affects cell viability. Thus examples may include antibiotic resistance genes such as neomycin. Particularly preferred examples include gene products which are toxic to cells, either directly or via the conversion of a prodrug into a toxic compound. An example of the latter is the enzyme thymidine kinase which converts compounds such as gancyclovir into cytotoxic compounds. Thus is it preferred that the second nucleotide sequence encodes a gene product that is capable, when expressed, of reducing cell viability, including by causing cell death, either directly or in the presence of an exogenously added compound.

An important feature of the second nucleotide construct is that in addition to the coding region, at least one third nucleotide sequence is present either upstream or downstream of the coding sequence, which third nucleotide sequence it is desired to test the library of inhibitory RNA molecules against. For example, the third nucleotide sequence may be a component of a virus such as a component of a retrovirus, such as a sequence encoding all or part of a gag.pol sequence. The presence of the third nucleotide sequence either upstream or downsteam of the detectable marker coding sequence means that in the event that one of the inhibitory RNA molecules encoded by the first nucleotide sequence binds to the third nucleotide sequence, or its transcription product, the resulting interaction prevents transcription of the second nucleotide sequence and/or causes a decrease in the stability of the mRNA produced from the second nucleotide sequence such that the expression of the detectable marker is reduced or inhibited. In the case of thymidine kinase, inhibition of TK expression removes the susceptibility of the cells to gancyclovir. Consequently, any surviving cells do not express sufficient TK to cause cell death in the presence of gancyclovir. When TK/gancyclovir are used, it is preferred to also administer dieldrin to minimise the bystander effect due to the cytotoxic compounds passing into neighbouring cells via gap junctions. Similar considerations apply to other cytotoxic compounds that have a bystander effect.

The third nucleotide sequence is therefore positioned relative to the coding sequence such that it is between the coding sequence and sequences required for mRNA stability and/or translation. For example, the third nucleotide sequence may be placed such that in the transcribed mRNA it is just downstream of the 5′ cap or just upstream of the 3′ polyA tail. An inhibitory RNA molecule which binds to and causes cleavage of the 5′ cap and/or the polyA tail will enhance degradation of the remainder of the mRNA. Thus, the third nucleotide sequence will typically be present within the 5′ and/or 3 ′ untranslated regions (UTRs) of the mRNA encoding the detectable marker.

The third nucleotide sequence may be any nucleotide sequence for which it is desired to identify inhibitory RNA molecules which function efficiently against it. Thus, it may encode all or part of a gene product whose expression it is desired to inhibit or reduce. Such gene products may include mammalian cell gene products whose expression is associated with disease. Alternatively, the third nucleotide sequence may encode a component of a pathogenic organism, particularly a viral pathogen such as a retroviral pathogen, including HIV. Such components may, for example include gag.pol or env or accessory factors.

As for the first nucleotide sequence, the second nucleotide sequence comprising one or more third nucleotide sequences, is typically part of a nucleic acid vector as discussed in section B.

B. Nucleic Acid Vectors

The first and second nucleotide sequences are typically present in nucleic acid vectors, which may be the same or different nucleic acid vectors operably linked to a regulatory control sequence which permits expression of the first and second nucleotide sequences in a host cell.

The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of the inhibitory RNA molecules. Vectors will be chosen that are compatible with the host cell used.

Control sequences operably linked to sequences encoding the inhibitory RNA molecules include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian cells, although promoters functional in other eukaryotic cells, such as insect cells, may be used. The promoter is typically derived from promoter sequences of viral or eukaryotic genes. For example, it may be a promoter derived from the genome of a cell in which expression is to occur. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

The vector used in the method of the present invention may be may be non-viral, for example, plasmids, chromosomes or artificial chromosomes. Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Alternatively, the vector may be a viral vector. Viral vectors include but are not limited to adenovirus vector, an adeno-associated viral (AAV) vector, a herpes viral vector, retroviral vector, lentiviral vector, baculoviral vector. The term “viral vector” refers to a nucleotide construct comprising a viral genome capable of being transcribed in a host cell, which genome comprises sufficient viral genetic information to allow packaging of the viral RNA genome, in the presence of packaging components, into a viral particle capable of infecting a target cell. Infection of the target cell includes reverse transcription and integration into the target cell genome, where appropriate for particular viruses. The viral vector in use typically carries heterologous coding sequences (nucleotides of interest) which are to be delivered by the vector to the target cell, for example a first nucleotide sequence encoding an inhibitory ribozyme. A viral vector is incapable of independent replication to produce infectious viral particles within the final target cell.

The term “viral vector system” is intended to mean a kit of parts which can be used when combined with other necessary components for viral particle production to produce viral particles in host cells. For example, the plurality of first nucleotide sequences may typically be present in a plasmid vector constructs suitable for cloning the first nucleotide sequences into a viral genome vector construct. When combined in a kit with a second nucleotide sequence (the reporter construct), which may be present in a separate plasmid vector construct the resulting combination of the plurality of plasmids containing the first nucleotide sequence and plasmid containing the second nucleotide sequence comprises the essential elements of the invention. Such a kit may then be used by the skilled person in the production of suitable viral vector genome constructs which when transfected into a host cell together with the plasmids and optionally nucleic acid constructs encoding other components required for viral assembly, will lead to the production of infectious viral particles.

The second nucleotide sequence may also conveniently be present as part of the same vector construct as the first nucleotide construct. In this way, co-expression of the first nucleotide sequence and the reporter construct (such as the TK gene) in any individual cell is ensured. Similarly, viral vectors may also comprise both the first and second nucleotide sequences.

Alternatively, the second nucleotide sequence may be stably present within a packaging cell line that is included in the kit.

The kit may include the other components needed to produce viral particles, such as host cells and other plasmids encoding essential viral polypeptides required for viral assembly. By way of example, the kit may contain a plurality of plasmids containing a first nucleotide sequence encoding an anti-HIV ribozyme and a second nucleotide sequence encoding TK operably linked to a third nucleotide sequence placed within the 5 ′ and/or 3′ UTR. Optional components would then be (a) an HIV viral genome construct with suitable restriction enzyme recognition sites for cloning the first and second nucleotide sequences into the viral genome; (b) a plasmid encoding a VSV-G env protein and (c) a plasmid encoding gag.pol. Alternatively, nucleotide sequence encoding viral polypeptides required for assembly of viral particles may be provided in the kit as packaging cell lines comprising the nucleotide sequences, for example a VSV-G expressing cell line.

Viral vectors are typically retroviral vectors, in particular lentiviral vectors such as HIV vectors. The retroviral vector of the present invention may be derived from or may be derivable from any suitable retrovirus. A large number of different retroviruses have been identified. Examples include: murine leukemia virus (MLV), human immunodeficiency virus (HIV), simian immunodeficiency virus, human T-cell leukemia virus (HTLV) equine infectious anaemia virus (EIAV), mouse mammary tumour virus (MMLV). Rous sarcoma virus (RSV), Fujinami sarcoma virus (FuSV), Moloney murine leukemia virus (Mo-MLV). FBR murine osteosarcoma virus (FBR MSV). Moloney murine sarcoma virus (Mo-MSV), Abelson murine leukemia virus (A-MLV). Avian myelocytomatosis virus-29 (MC29), and Avian erythroblastosis virus (AEV). A detailed list of retroviruses may be found in Coffin et al., 1997. “Retroviruses”. Cold Spring Harbour Laboratory Press Eds: J M Coffin. S M Hughes. H E Varmus pp 758-763.

Details on the genomic structure of some retroviruses may be found in the art. By way of example, details on HIV and Mo-MLV may be found from the NCBI Genbank (Genome Accession Nos. AF033819 and AF033811. respectively).

The lentivirus group can be split even further into “primate” and “non-primate”. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3. R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional. The R regions at both ends of the RNA are repeated sequences. U5 and U3 represent unique sequences at the 5′ and 3′ ends of the RNA genome respectively.

In a typical retroviral vector for use in gene therapy and/or other techniques of molecular biology, at least part of one or more of the gag, pol and env protein coding regions essential for replication may be removed from the virus. This makes the retroviral vector replication-defective. The removed portions may even be replaced by a nucleotide sequence of interest (NOI), such as a first nucleotide, to generate a virus capable of integrating its genome into a host genome but wherein the modified viral genome is unable to propagate itself due to a lack of structural proteins. When integrated in the host genome, expression of the NOI occurs—resulting in, for example, a therapeutic and/or a diagnostic effect. Thus, the transfer of an NOI into a site of interest is typically achieved by: integrating the NOI into the recombinant viral vector; packaging the modified viral vector into a virion coat; and allowing transduction of a site of interest—such as a targeted cell or a targeted cell population.

A minimal retroviral genome for use in the present invention will therefore comprise (5′) R—U5 —a first nucleotide sequence and/or a second nucleotide sequence—U3-R (3′). However, the plasmid vector used to produce the retroviral genome within a host cell/packaging cell will also include transcriptional regulatory control sequences operably linked to the retroviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter.

Some retroviral genomes require additional sequences for efficient virus production. For example, in the case of HIV, rev and RRE sequence are preferably included. However the requirement for rev and RRE can be reduced or eliminated by codon optimisation.

Once the retroviral vector genome is integrated into the genome of its target cell as proviral DNA, the ribozyme sequences need to be expressed. In a retrovirus, the promoter is located in the 5′ LTR U3 region of the provirus. In retroviral vectors, the promoter driving expression of a heterologous gene may be the native retroviral promoter in the 5′ U3 region, or an alternative promoter engineered into the vector. The alternative promoter may physically replace the 5′ U3 promoter native to the retrovirus, or it may be incorporated at a different place within the vector genome such as between the LTRs.

Replication-defective retroviral vectors are typically propagated, for example to prepare suitable titres of the retroviral vector for subsequent transduction, by using a combination of a packaging or helper cell line and the recombinant vector. That is to say, that the three packaging proteins can be provided in trans.

A “packaging cell line” contains one or more of the retroviral gag, pol and env genes. The packaging cell line produces the proteins required for packaging retroviral DNA but it cannot bring about encapsidation due to the lack of a psi region. However, when a recombinant vector carrying an NOI and a psi region is introduced into the packaging cell line, the helper proteins can package the psi-positive recombinant vector to produce the recombinant virus stock. This virus stock can be used to transduce cells to introduce the NOI into the genome of the target cells. It is preferred to use a psi packaging signal, called psi plus, that contains additional sequences spanning from upstream of the splice donor to downstream of the gag start codon (Bender et al., 1987) since this has been shown to increase viral titres.

The recombinant virus whose genome lacks all genes required to make viral proteins can tranduce only once and cannot propagate. These viral vectors which are only capable of a single round of transduction of target cells are known as replication defective vectors. Hence, the NOI is introduced into the host/target cell genome without the generation of potentially harmful retrovirus. A summary of the available packaging lines is presented in Coffin et al., 1997 (ibid).

Retroviral packaging cell lines in which the gag pol and env viral coding regions are carried on separate expression plasmids that are independently transfected into a packaging cell line are preferably used. This strategy, sometimes referred to as the three plasmid transfection method (Soneoka et al., 1995) reduces the potential for production of a replication-competent virus since three recombinant events are required for wild type viral production. As recombination is greatly facilitated by homology, reducing or eliminating homology between the genomes of the vector and the helper can also be used to reduce the problem of replication-competent helper virus production.

An alternative to stably transfected packaging cell lines is to use transiently transfected cell lines. Transient transfections may advantageously be used to measure levels of vector production when vectors are being developed. In this regard, transient transfection avoids the longer time required to generate stable vector-producing cell lines and may also be used if the vector or retroviral packaging components are toxic to cells. Components typically used to generate retroviral vectors include a plasmid encoding the gag/pol proteins, a plasmid encoding the env protein and a plasmid containing an NOI. Vector production involves transient transfection of one or more of these components into cells containing the other required components. If the vector encodes toxic genes or genes that interfere with the replication of the host cell, such as inhibitors of the cell cycle or genes that induce apotosis, it may be difficult to generate stable vector-producing cell lines, but transient transfection can be used to produce the vector before the cells die. Also, cell lines have been developed using transient transfection that produce vector titre levels that are comparable to the levels obtained from stable vector-producing cell lines.

Producer cells/packaging cells can be of any suitable cell type. Most commonly, mammalian producer cells are used but other cells, such as insect cells are not excluded. Clearly, the producer cells will need to be capable of efficiently translating the env and gag, pol mRNA. Many suitable producer/packaging cell lines are known in the art. The skilled person is also capable of making suitable packaging cell lines by, for example stably introducing a nucleotide construct encoding a packaging component into a cell line.

Where the first nucleotide sequence within the retroviral genome encodes an inhibitory RNA molecule which is being tested for it ability to effect the cleavage of gag, pol and/or env RENA transcripts the nucleotide sequences present in the packaging cell line, either integrated or carried on plasmids, or in the transiently transfected producer cell line, which encode gag, pol and or env proteins may be modified so as to reduce or prevent binding of the inhibitory RNA molecule(s). In this way, the inhibitory RNA molecule(s) will not prevent expression of viral polypeptides in packaging cell lines that are essential for packaging of viral particles. Clearly, given the random nature of the library, it is still possible that some sequences within the library will cleave modified packaging components but by having the third nucleotide sequence and the sequence encoding the corresponding packaging component different, an inhibitory molecule which affect the modified packaging component is less likely also simultaneously to affect the third nucleotide sequence and therefore the cell will in many cases produce the detectable marker and be eliminated from the screen.

The term “viral polypeptide essential for packaging of viral particles” means a polypeptide normally encoded by the viral genome to be packaged into viral particles, in the absence of which the viral genome cannot be packaged. For example, in the context of retroviruses such polypeptides would include gag, pol and env. The terms “packaging component” and “essential component” are also included within this definition.

By way of example, in the case of ribozymes resistance is typically by virtue of alterations in the sequences which eliminate the ribozyme recognition sites. At the same time, the amino acid coding sequence for the essential/packaging components is retained so that the viral components encoded by the sequences remain the same, or at least sufficiently similar that the function of the essential/packaging components is not compromised. Again, given the often random nature of the plurality of first nucleotide sequences, extensive modification of the nucleotide sequence encoding the packing component may be required.

In the case of antisense sequences, the third nucleotide sequence differs from the nucleotide sequence encoding the viral packaging component to the extent that although an antisense sequence can bind to the third nucleotide sequence, or transcript thereof, the antisense sequence can not bind effectively to the nucleotide sequence encoding the required packaging component or RNA transcribed from therefrom. The differences between the third nucleotide sequences and the nucleotide sequences encoding a required packaging component will typically be conservative changes, although a small number of amino acid changes may be tolerated provided that, as described above, the function of the essential/packaging components is not significantly impaired.

More generally, a suitable approach may be to alter the nucleotide sequence of the required packaging component so that the nucleotide homology over the region corresponding to the third nucleotide sequence is less than 95%, preferably less than 90, 80 or 70% whilst the amino acid homology is preferably at least 95%.

Preferably, in addition to eliminating the inhibitory RNA recognition sites, the alterations to the coding sequences for the viral components improve the sequences for codon usage in the mammalian cells or other cells which are to act as the producer cells for retroviral vector particle production. This improvement in codon usage is referred to as “codon optimisation”. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Thus preferably, the sequences encoding the packaging components are codon optimised. More preferably, the sequences are codon optimised in their entirety. Following codon optimisation, it is found that there are numerous sites in the wild type gag, pol and env sequences which can serve as inhibitory RNA recognition sites and which are no longer present in the sequences encoding the packaging components.

An additional advantage of codon optimising HIV packaging components is that this can increase gene expression. In particular it can render gag, pol expression Rev independent so that rev and RRE need not be included in the genome (Haas et al., 1996). Rev-independent vectors are therefore possible. This in turn enables the use of anti-rev or RRE factors in the retroviral vector.

It is highly desirable to use high-titre virus preparations in both experimental and practical applications. Techniques for increasing viral titre include using a psi plus packaging signal as discussed above and concentration of viral stocks. In addition, the use of different envelope proteins, such as the G protein from vesicular-stomatitis virus has improved titres following concentration to 10⁹ per ml. However, typically the envelope protein will be chosen such that the viral particle will preferentially infect cells that are infected with the virus which it desired to treat. For example where an HIV vector is being used to treat HIV infection, the env protein used will be the HIV env protein.

C. Selection Methods

The selection method of the invention involves introducing one or more first nucleotide sequences into a host cell in the presence of a second nucleotide sequence. Methods for introducing nucleic acids into host cells are well known in the art, for example transfection. Generally, the nucleotide sequences are part of a vector. The first and second nucleotides sequences may be part of the same vector or present on separate nucleic acid molecules. The second nucleotide sequence may even be stably introduced into the genome of the host cell.

If an inhibitory RNA molecule encoded by a first nucleotide sequence inhibits or reduces expression of a detectable marker then it will be possible to distinguish cells containing such a molecule from cells that contain a first nucleotide sequence that does not lead to an inhibition in or reduction of expression of the detectable marker. Cells that contain the effective inhibitory RNA molecule can be used in, for example, a PCR reaction, to identify the sequence of the inhibitory RNA molecule and thus the sequence of the region of the target molecule.

If the detectable marker is FACS-sortable, then the cells which contain an effective inhibitory RNA molecule can be sorted from the remainder of the cells. If the selectable marker is an enzyme capable of converting a prodrug into a cytotoxic compound, those cells which contain an effective inhibitory RNA molecule will be selectable based on their insensitivity to the prodrug. More specifically, those cells which contain an effective inhibitory RNA molecule will be better able to survive selection with the prodrug.

In a preferred embodiment, the latter selection process is conveniently performed using a library of viral vectors comprising first nucleotide sequences. The viral vector library is transfected into producer cells comprising the second nucleotide sequence encodings for example. TK. Cells which survive subsequent prodrug (for example, gancyclovir) selection will generally comprise at least one first nucleotide sequence encoding an effective inhibitory RNA molecule. A further optional stage is to harvest the viral particles from said cells and use the infectious viral particles to transduce further cells. An advantage of this extra step is that, typically, only first nucleotide sequences that do not cleave essential viral sequences/packaging components will be carried through successfully to this second stage. In addition, by making a dilution series of the viral supernatant obtained from the viable cells, it may be possible to produce cells comprising only one first inhibitory sequence which will simplify identification by PCR.

It should be noted that some inhibitory molecules may cause cell death for other reasons other than by failure to target a third nucleotide sequence. For example an inhibitory molecule may cause degradation of an mRNA essential for cell survival. In general, since cells containing such molecules will die, selection against these elements of a random library is not required.

“False positives” may also be obtained if the first nucleotide sequence is capable of binding to and effecting the cleavage of the coding region encoding the detectable marker. For example, if the selection process depends on the capacity of those cells which contain an effective inhibitory RNA molecule to survive selection with a prodrug, false positives may be obtained if the inhibitory molecule(s) directly target the mRNA encoding the enzyme which converts the prodrug into a cytotoxic compound. In an especially preferred embodiment, a selection step is included to eliminate vectors encoding inhibitory molecules that target sequences of the detectable marker used in the second nucleic acid.

This can be done, for example, by inserting the first nucleotide sequences into a construct comprising a non-functional nucleotide sequence encoding a detectable marker and lacking the third nucleotide sequence (“a fourth nucleotide sequence”). Preferably, the other vector sequences are identical to those of the vector comprising the second nucleotide sequence. Typically, the nucleotide sequence encoding the detectable marker has been modified by insertion or deletion to prevent expression of a functional detectable marker. The inactive detectable marker is thus the same sequence as detectable marker sequence used in the second nucleic acid, apart from the mutation which renders it non-functional.

The construct can then be used to generate a library viral vectors for the initial screening stage. The genomes encoding inhibitory molecules that (i) are capable of binding to and causing cleavage of the detectable marker; (ii) affect the viral genome in which they are contained; (iii) affect viral packaging; or (iv) kill cells, will not be packaged into viral particles. Thus a pool of viruses may be produced that comprise a plurality of first nucleotide sequences preselected so as not to be (i) capable of causing cleavage of the detectable marker; (ii) detrimental to cell survival, or (iii) capable of inhibiting viral production in the selection assay.

This pool of viruses can then be used to transduce cells comprising the second nucleotide sequence in a selection round as described above.

D. Uses of Optimised Inhibitory RNA Molecules

Optimised inhibitory RNA molecules, such as ribozymes, identified by the selection method of the invention, may be used to inhibit expression of their target nucleotide sequence or transcription product thereof in a target cell.

This may be used to ablate the expression of genes in cells in vitro or in vivo in order to investigate the effect of blocking gene expression, for example by conducting “knock-out” experiments. Such experiments are useful, for example, for target validation, screening or model building.

Alternatively, optimised inhibitory RNA molecules identified by the selection method of the invention may be used in therapeutic applications such as gene therapy and direct administration of RNA. In particular, optimised inhibitory RNA molecules which target nucleotide sequence encoding essential components of pathogens may be used to treat disease caused by said pathogens. Thus an optimised inhibitory RNA molecule that targets a component of HIV may be used to reduce or prevent an HIV infection or associated symptoms.

Thus nucleic acid vectors comprising nucleotide sequences encoding optimised inhibitory RNA molecules may be used to treat or prevent viral infections, preferably retroviral infections, in particular lentiviral, especially HIV, infections. Specifically, said nucleic acid vectors may be used to deliver inhibitory RNA molecules to a human or animal in need of treatment for a viral infection. Nucleic acid vectors include viral vectors and infectious viral particles obtained therefrom. Where the viral vector comprises a selected first nucleotide sequence which targets a component of a pathogenic virus, which component is also required for the generation of infectious viral particles comprising the selected first nucleotide sequence, the nucleotide sequence of the component in packaging/producer cells will typically be modified as described above. In particular, guidance is given in the reference examples with respect to the use of ribozymes and modified retroviral env and gag.pol sequences.

HIV-1 provides an ideal vector for the development of strategies to combat HIV-1 infection. This is because a genome with the open reading frames removed and packaged by trans-produced viral particles (hence non-pathogenic, but still immunogenic) can be used to carry anti-HIV molecules such as ribozymes, intra-bodies, intra-kines, RNA decoys and/or trans-dominant mutated proteins.

If the splice donor and splice acceptor sites are retained then up to 8 distinct anti-HIV factors could be generated by one viral construct. For example the Tat reading frame could be replaced with a ribozyme targeting Tat and the nef reading frame be replaced with a tat transdominant mutant.

Inhibitory RNA molecules such as ribozymes would be a key component of such a system.

Preferably the nucleic acid vectors/viral particles are combined with a pharmaceutically acceptable carrier or diluent to produce a pharmaceutical composition. Thus, the present invention also provides a pharmaceutical composition for treating an individual, wherein the composition comprises a therapeutically effective amount of the nucleic acid vector/viral particle of the present invention, together with a pharmaceutically acceptable carrier, diluent, excipient or adjuvant. The pharmaceutical composition may be for human or animal usage.

The choice of pharmaceutical carrier, excipient or diluent can be selected with regard to the intended route of administration and standard pharmaceutical practice. Suitable carriers and diluents include isotonic saline solutions, for example phosphate-buffered saline. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

The pharmaceutical composition may be formulated for parenteral, intramuscular, intravenous, intracranial, subcutaneous, intraocular or transdermal administration.

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The amount of virus administered is typically in the range of from 10³ to 10¹⁰ pfu, preferably from 10⁵ to 10⁸ pfu, more preferably from 10⁶ to 10⁷ pfu. When injected, typically 1-10 μl of virus in a pharmaceutically acceptable suitable carrier or diluent is administered.

When the polynucleotide/vector is administered as a naked nucleic acid, the amount of nucleic acid administered is typically in the range of from 1 μg to 10 mg, preferably from 100 μg to 1 mg.

Where a first nucleotide sequence selected by the method of the invention (or other therapeutic sequence) is under the control of an inducible regulatory sequence it may only be necessary to induce gene expression for the duration of the treatment. Once the condition has been treated, the inducer is removed and expression of the nucleotide sequence is stopped. This will clearly have clinical advantages. Such a system may, for example, involve administering the antibiotic tetracycline, to activate gene expression via its effect on the tet repressor/VP16 fusion protein.

E. In vivo Selection Methods

In one aspect, the present invention provides a method for identifying an open site on a mRNA molecule.

The term “open” means a site which, when the mRNA is in its physiological confirmation, is accessible to inhibitory RNA molecules such as ribozymes, antisense RNA or EGSs.

Using the selection system of the present invention, the mRNA is encoded by the third nucleotide sequence. For example, if the third nucleotide sequence is a whole gene, the selection system can be used to identify one or more open sites on the corresponding mRNA molecule. Once an inhibitory RNA molecule (such as ribozymes, antisense RNA or EGSs) has been identified as capable of binding to the third nucleotide sequence using the selection system, the target site can be deduced using standard techniques. For example, if the inhibitory molecule is an antisense sequence, a sequence comparison between the “optimum” complementary sequence of the antisense sequence, and the mRNA sequence should reveal the site at which the antisense sequence bound to the mRNA.

The invention will now be further described by way of Examples, which are meant to serve to assist one of ordinary skill in the art in carrying out the invention and are not intended in any way to limit the scope of the invention. The Examples refer to the Figures. In the

### FIGURES

FIG. 1. Prodrug dose response—Graph of percentage survival vs prodrug concentration for: A. E. coli Nitroreductase (NTR)/Metronidazole (MTZ) and B. HSV-1 Thymidine kinase (TK)/Gancyclovir (GCV).

FIG. 2. Bystander Effect—Graph of percentage survival vs prodrug concentration for A. NTR/MTZ and B. TK/GCV

FIG. 3 shows a bar chart of % survival for HeLa cell lines comprising CTKtatSN which have been transduced with pH4Z (tat-4Z), an anti-tat ribozyme with the catalytic activity removed (tat-RzM) or an anti-tat ribozyme (tat-Rz). The cells have been incubated with GCV or GCV and dieldrin.

FIG. 4 shows a schematic representation of vector genomes.

FIG. 5 shows schematically ribozymes inserted into four different HIV vectors;

FIG. 6 shows schematically how to create a suitable 3 ′ LTR by PCR;

FIG. 7 shows the codon usage table for wild type HIV gag.pol of strain HXB2 (accession number: K03455).

FIG. 8 shows the codon usage table of the codon optimised sequence designated gag,pol-SYNgp.

FIG. 9 shows the codon usage table of the wild type HIV env called env-mn.

FIG. 10 shows the codon usage table of the codon optimised sequence of HIV env designated SYNgp160 mn.

FIG. 11 shows three plasmid constructs for use in the invention.

FIG. 12 shows the principle behind two systems for producing retroviral vector particles.

FIGS. 13 A-B—design of external guide sequences. (SEQ ID NOs: 22-26)

The invention will now be further described in the Examples which follow, which are intended as an illustration only and do not limit the scope of the invention.

### EXAMPLES

The present inventors have developed a method which is based on the generation of a library of ribozymes each containing the catalytic domain of a hammerhead ribozyme. The procedure involves generating a virus that contains a genome comprising a nucleotide sequence that encodes a suicide gene (thymidine kinase (TK) is preferred). The target sequence is inserted downstream of this. Alternatively, or in addition, if an internal ribosome entry site (IRES) is inserted upstream of the TK sequence then the target sequence can be inserted upstream. Cells that are transduced with this vector will be sensitive to gancyclovir.

However, when the viral genome is transcribed the mRNA contains not only the TK gene but also the target sequence. This mRNA will contain the normal 5′ cap and the 3′ poly A tail. If the 5′ cap or the poly A tail are removed by cleavage with a ribozyme the mRNA is unstable and is degraded. This prevents the translation of the TK gene, rendering cells that express an active ribozyme insensitive to gancyclovir.

### Example 1

### Selection of Prodrug System (Selectable Marker)

The second nucleotide used in the present invention encodes a detectable marker. We have chosen to exemplify the present invention using a enzyme-prodrug selection system. The dose response of two well-known enzyme-prodrug combinations was measured to select an effective prodrug system.

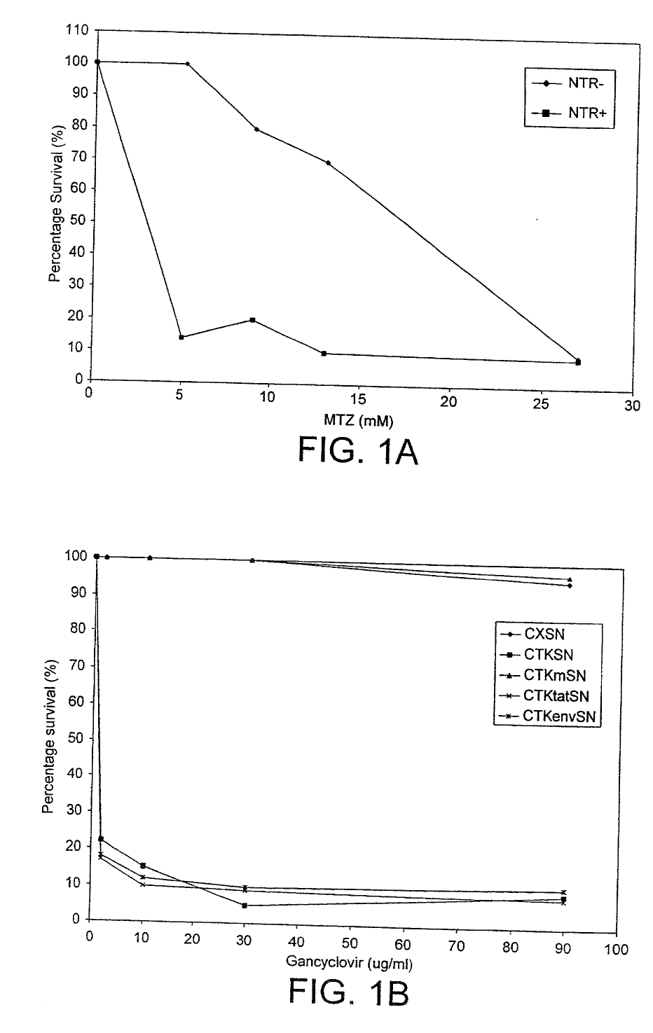

A. An E. coli Nitroreductase (NTR)/Metronidazole (MTZ) combination: HT1080 cells (NTR−) and HT1080 NTR positive cells (NTR+) were incubated with varying concentrations of MTZ and the percentage of cells which survived was measured (FIG. 1A).

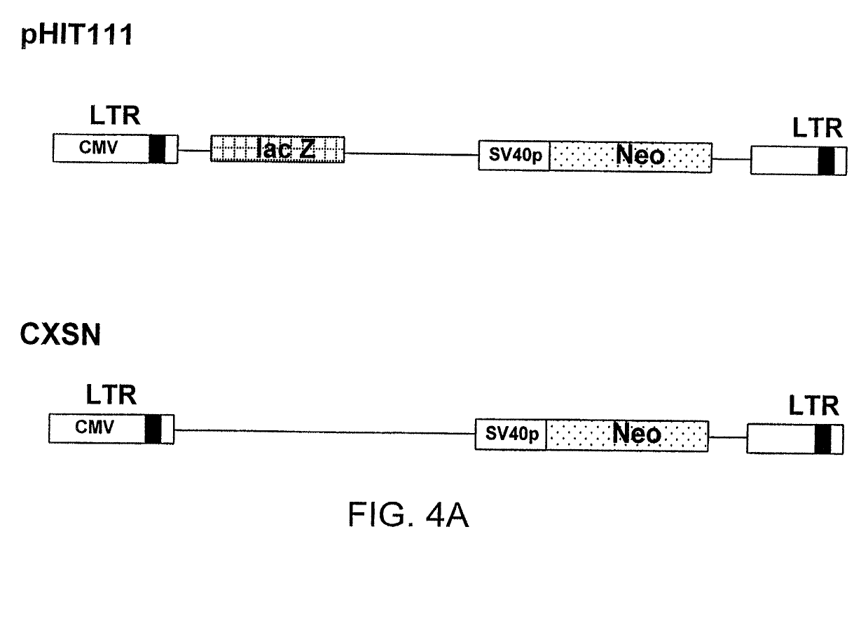

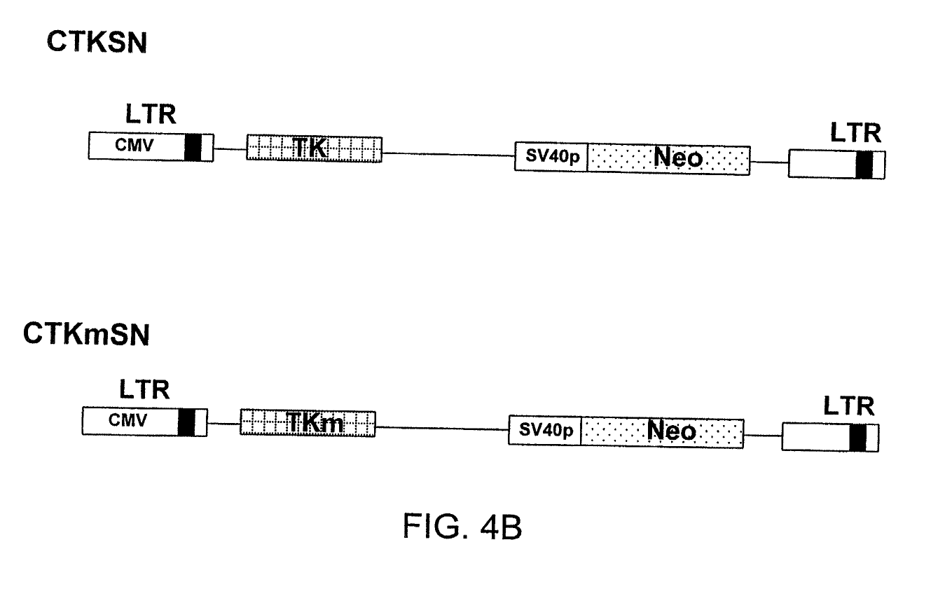

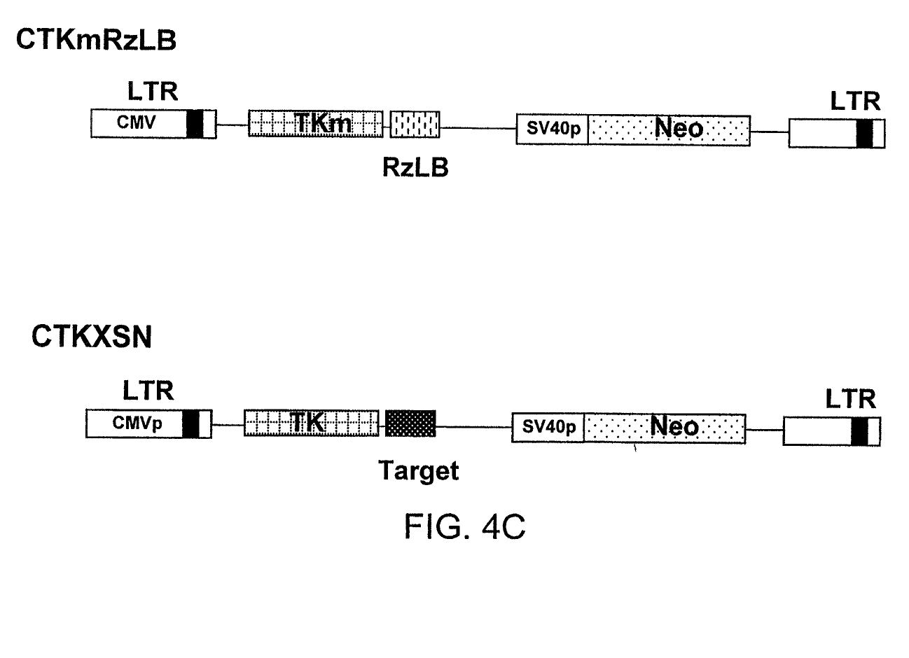

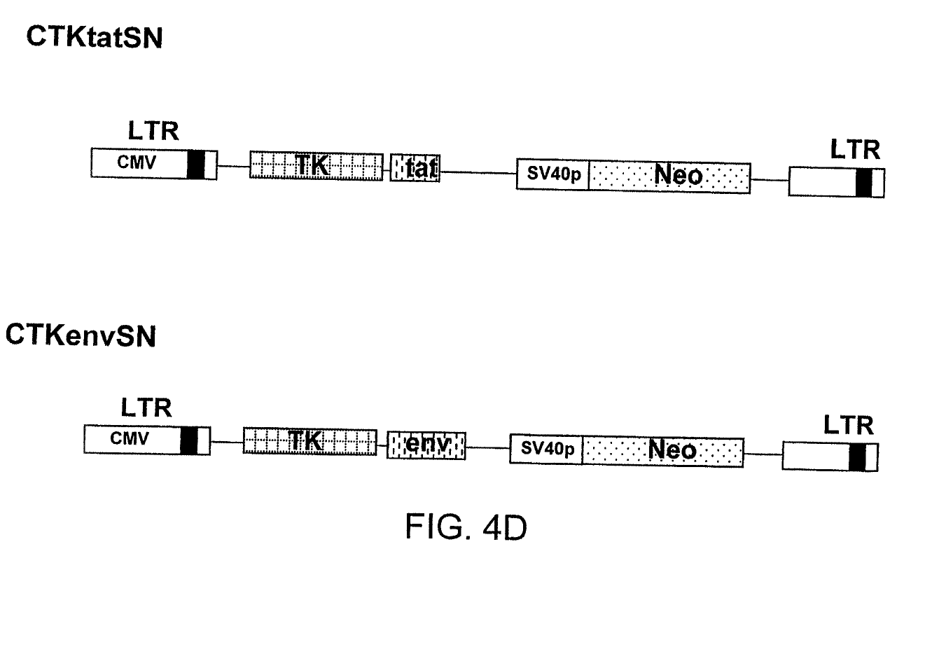

B. An HSV-1 Thymidine kinase (TK)/Gancyclovir (GCV) combination: HeLa cell lines transduced with various viral vectors were treated for 72 hours with GCV and the percentage of cells which survived was measured (see FIG. 1B). CXSN=empty viral vector, CTKSN=TK positive virus, CTKmSN=TK mutant virus, CTKtatSN=vector containing the HIV-1 tat gene ORF downstream of an active TK gene (FIG. 4D), CTKenvSN=vector containing the HIV-1 env gene ORF downstream of an active TK gene (FIG. 4D). CXSN is made by removing the lac Z gene from pHIT111. C=CMV promoter in place of the 5′ U3, X=cloning site, for the gene of interest, S=SV40 promoter and N=Neomycin resistance gene. Tk=thymidine kinase, TKm=thymidine kinase mutant.

These results indicate that better sensitivity can be obtained with a TK/GCV system. However, TK/GCV produces a bystander effect. Generally, the drug used should not have a bystander effect, i.e. cause the death of cells surrounding the cell that is expressing the enzyme that activates the prodrug. The bystander effect is due to the active form of the prodrug either crossing the plasma membrane and entering surrounding cells or entering other cells through gap junctions. In the case of GCV the bystander effect is caused by the drug crossing through gap junctions. Fortunately, these gap junctions can be blocked with the drug dieldrin and hence limit the bystander effect (Touraine et al., 1998).

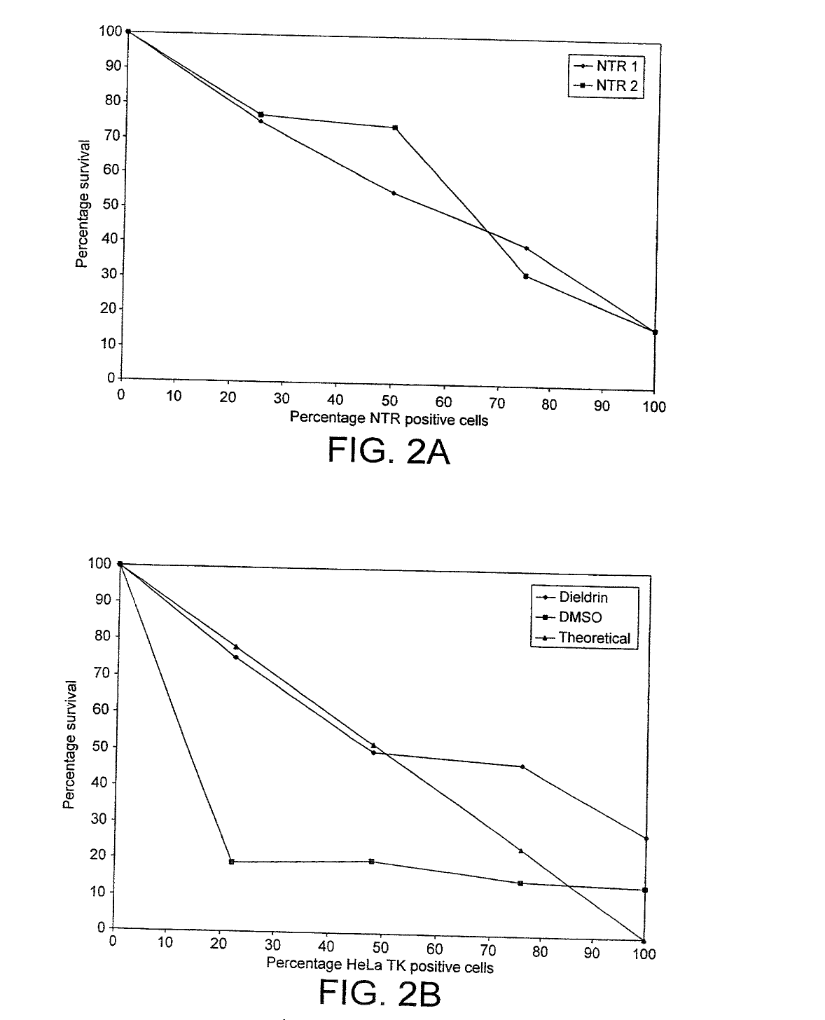

FIG. 2 shows the bystander effect of the two enzyme/prodrug systems mentioned above. In FIG. 2A, an HT1080 NTR positive stable cell line was co-cultured with HT1080 cells in the presence of 10 mM MTZ for 24 hours. In FIG. 2B, a HeLa TK stable cell line was co-cultured with HeLa cell with GCV in the presence of absence of dieldrin (16 μg/1 ml) for 72 hours.

From this data it is clear that the TK/GCV bystander effect can be minimised using dieldrin.

Thus, we have chosen to use HSV-1 TK/GCV/dieldrin, in preference to NTR/metronidazole (MTZ) which does not have a bystander effect, because TK/GCV is more sensitive in distinguishing cells with or without the enzyme (FIGS. 1A. 1B). The amount of GCV used in the study is 3 μg/ml as determined from FIG. 1.

### Example 2

### In vivo Selection Using Hammerhead Ribozymes

A library of hammerhead ribozymes is generated by random oligonucleotide synthesis. These are then inserted into CTKmSN (FIG. 4B) downstream of the inactive TK coding sequence to give a plurality of enzymes (CTKmRzLB—FIG. 4C).

CTKmSN (FIG. 4B) was constructed by removing the lacZ of pHIT111 (Soneoka et al. 1995) (FIG. 4A) to give the intermediate construct CKSN (FIG. 4A) and then inserting in its place an inactive TK coding sequence constructed by a frameshift mutation (cut and re-fill at BsP EI site). The sequence of human herpes virus 1 is provided in Wagner et al., 1981 (Genbank accession no. V00467).

Insertion of the library downstream of the inactive form of the enzyme is necessary to ensure that no ribozymes that cut the TK containing RNA (and therefore would lead to false positives in the selection) are contained in the vector library. Virus was generated by the three plasmid co-transfection system (Soneoka et al., 1995) and used to transduce HeLa cells. The activity of the mutant was assessed by measuring cell survival after exposure to GCV.

The target RNA sequences are inserted in the viral vector CTKSN (FIG. 4B) downstream of the active form of the enzyme, to make CTKXSN where X=target sequence (FIG. 4C). CTKSN is made by inserting the TK sequence in CXSN (FIG. 4A). Following three plasmid co-tranfection the virus is used to transduce cells such as NIH3T3. HeLa or any other cells. HeLa cells were chosen in order to minimise bystander effects (HeLa cells have a low percentage of gap-junctional communication and the bystander effect of GCV is due to transmission of the monophosphate guanosine analog through gap junctions). Dieldrin was also used to minimise the bystander effect.

Stable cell lines are generated by neomycin selection (G418, 1 mg/ml for 10 days). These cell lines can now be transduced with the vector containing the ribozyme library (CTKmRxLB) and GCV selection will follow. Any ribozymes that cut the HIV sequence will prevent translation of TK and hence the cells will survive. All of the other cells will die due to retaining functional TK. PCR can then be used to establish the ribozyme sequence.

### Example 3

### In vivo Testing Using Optimised Ribozymes

The optimised ribosomes obtained according to Example 1 may be used in the treatment of diseases. In particular, a number of these ribozymes may be used in tandem allowing the targeting of a number of sites. In addition, in the treatment of HIV, an HIV vector can be used to deliver the ribozymes, as this will cause interference with the packaging of the wild type genome.

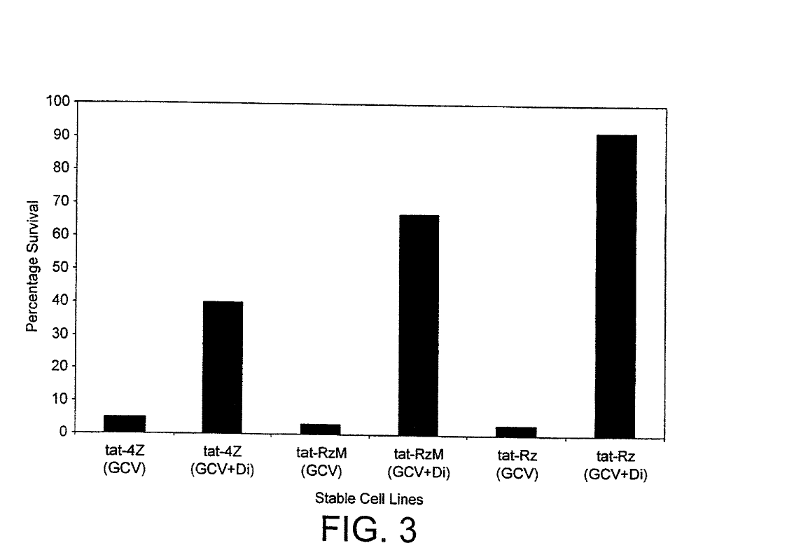

To test the feasibility of this approach we tested an anti-tat ribozyme on a tat stable cell line. As shown in FIG. 3, it is possible to select for cells that contain the functional ribozyme. It is also clear that the bystander effect has to be eliminated to be able to perform such a selection.

This technique could be used to find ribozymes specific for all parts of the HIV genome. In addition this method provides a means to isolate in vivo relevant ribozymes for any RNA target.

### Reference Example 1

### Construction of a Genome Carrying a Ribozyme

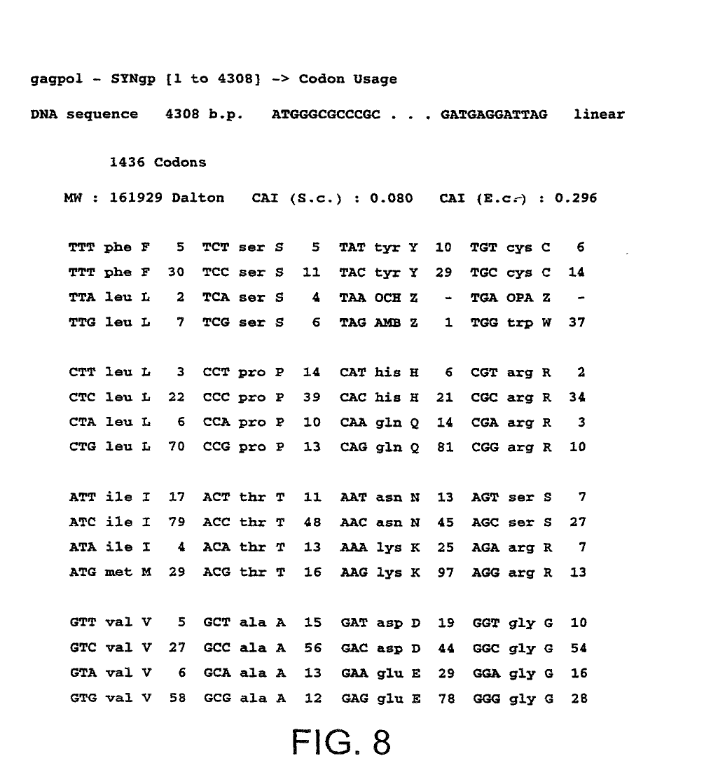

The HIV gag.pol sequence was codon optimised (FIG. 8 and SEQ I.D. No. 1) and synthesised using overlapping oligos of around 40 nucleotides. This has three advantages. Firstly it allows an HIV based vector to carry ribozymes and other therapeutic factors. Secondly the codon optimisation generates a higher vector titre due to a higher level of gene expression. Thirdly gag.pol expression becomes rev independent which allows the use of anti-rev or RRE factors.

Conserved sequences within gag.pol were identified by reference to the HIV Sequence database at Los Alamos National Laboratory (http://hiv-web.lanl.gov/) and used to design ribozymes. Because of the variability between subtype of HIV-1 the ribozymes were designed to cleave the predominant subtype within North America. Latin America and the Caribbean, Europe. Japan and Australia; that is subtype B. The sites chosen were cross-referenced with the synthetic gagpol sequence to ensure that there was a low possibility of cutting the codon optimised gagpol mRNA. The ribozymes were designed with XhoI and

SalI sites at the 5′ and 3 ′ end respectively. This allows the construction of separate and tandem ribozymes.

The ribozymes are hammerhead structures of the following general structure:

| Helix I | Helix II | Helix III |
|---|---|---|
| 5′-NNNNNNNN˜ | CUGAUGAGGCCGAAAGGCCGAA | ˜NNNNNNNN˜ |
| | (SEQ ID NO: 5) | |

The catalytic domain of the ribozyme (Helix II) can tolerate some changes without reducing catalytic turnover.

The cleavage sites, targeting gag and pol, with the essential GUX triplet (where X is any nucleotide base) are as follows:

GAG 1 5 ′ UAGUAAGAAUGUAUAGCCCUAC (SEQ ID NO:6)

GAG 2 5 ′ AACCCAGAUUGUAAGACUAUUU (SEQ ID NO:7)

GAG 3 5 ′ UGUUUCAAUUGUGGCAAAGAAG (SEQ ID NO:8)

GAG 4 5 ′ AAAAAGGGCUGUUGGAAAUGUG (SEQ ID NO:9)

POL 1 5 ′ ACGACCCCUCGUCACAAUAAAG (SEQ ID NO:10)

POL 2 5 ′ GGAAUUGGAGGUUUUAUCAAAG (SEQ ID NO:11)

POL 3 5 ′ AUAUUUUUCAGUUCCCUUAGAU (SEQ ID NO:12)

POL 4 5 ′ UGGAUGAUUUGUAUGUAGGAUC (SEQ ID NO:13)

POL 5 5 ′ CUUUGGAUGGGUUAUGAACUCC (SEQ ID NO:14)

POL 6 5 ′ CAGCUGGACUGUCAAUGACAUA (SEQ ID NO:15)

POL 7 5 ′ AACUUUCUAUGUAGAUGGGGCA (SEQ ID NO:16)

POL 8 5 ′ AAGGCCGCCUGUUGGUGGGCAG (SEQ ID NO:17)

POL 9 5 ′ UAAGACAGCAGUACAAAUGGCA (SEQ ID NO:18)

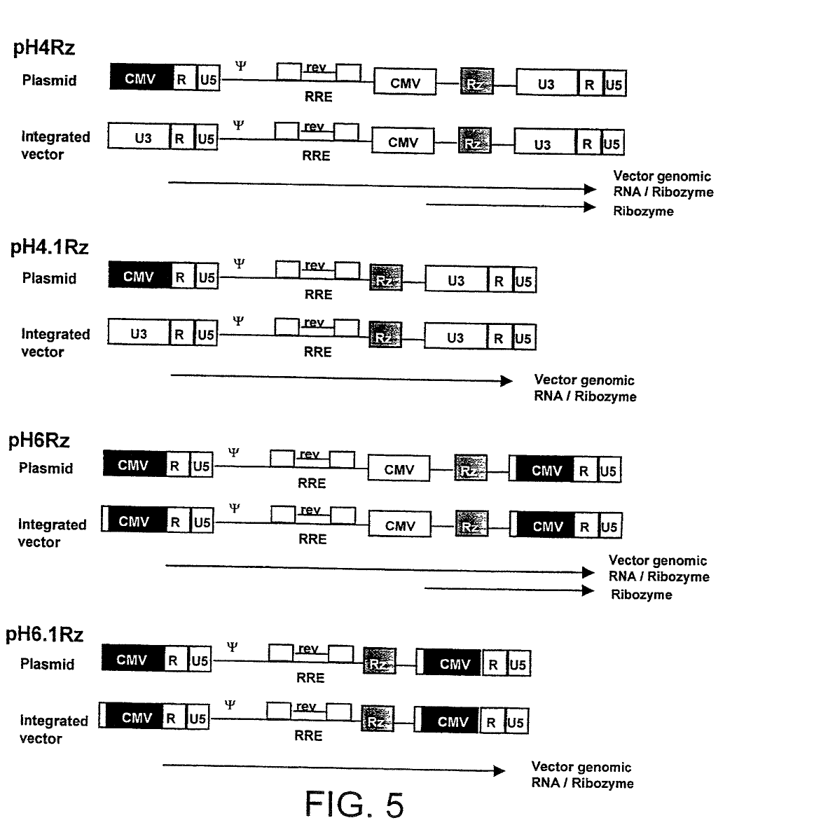

The ribozymes are inserted into four different HIV vectors (pH4 (Gervaix et al. 1997). pH6. pH4.1 or pH6.1) (FIG. 5). In pH4 and pH6. transcription of the ribozymes is driven by an internal HCMV promoter (Foecking and Hofstetter, 1986). From pH4.1 and pH6.1. the ribozymes are expressed from the 5′ LTR. The major difference between pH4 and pH6 (and pH4.1 and pH6.1) resides in the 3′ LTR in the production plasmid. pH4 and pH4.1 have the HIV U3 in the 3′ LTR. pH6 and pH6.1 have HCMV in the 3′ LTR. The HCMV promoter replaces most of the U3 and will drive expression at high constitutive levels while the HIV-1 U3 will support a high level of expression only in the presence of Tat.

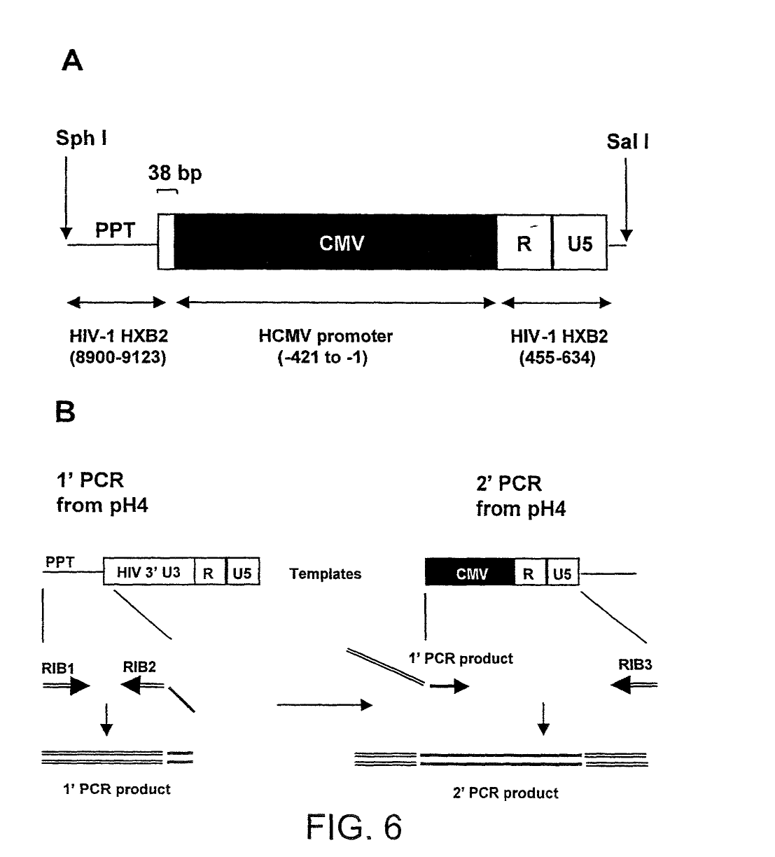

The HCMV/HIV-1 hybrid 3′ LTR is created by recombinant PCR with three PCR primers (FIG. 6). The first round of PCR is performed with RIB1 and RIB2 using pH4 (Kim et al., 1998) as the template to amplify the HIV-1 HXB2 sequence 8900-9123. The second round of PCR makes the junction between the 5′ end of the HIV-1 U3 and the HCMV promoter by amplifying the hybrid 5′ LTR from pH4. The PCR product from the first PCR reaction and RIB3 serves as the 5′ primer and 3′ primer respectively.

RIB1: 5′-CAGCTGCTCGAGCAGCTGAAGCTTGCATGC-3′ (SEQ ID NO:19)

RIB2: 5′-GTAAGTTATGTAACGGACGATATCTTGTCTTCTT-3′ (SEQ ID NO:20)

RIB3: 5′-CGCATAGTCGACGGGCCCGCCACTGCTAGAGATTTTC-3′ (SEQ ID NO:21)

The PCR product is then cut with SphI and SalI and inserted into pH4 thereby replacing the 3′ LTR. The resulting plasmid is designated pH6. To construct pH4.1 and pH6.1, the internal HCMV promoter (SpeI-XhoI) in pH4 and pH6 is replaced with the polycloning site of pBluescript II KS+(Stratagene) (SpeI-XhoI).

The ribozymes are inserted into the XhoI sites in the genome vector backbones. Any ribozymes in any configuration could be used in a similar way.

### Reference Example 2

### Construction of a Packaging System

The packaging system can take various forms. In a first form of packaging system, the HIV gag, pol components are co-expressed with the HIV env coding sequence. In this case, both the gag, pol and the env coding sequences are altered such that they are resistant to the anti-HIV ribozymes that are built into the genome. At the same time as altering the codon usage to achieve resistance, the codons can be chosen to match the usage pattern of the most highly expressed mammalian genes. This dramatically increases expression levels (Schneider et al., 1997; Schwartz et al., 1992) and so increases titre. A codon optimised HIV env coding sequence has been described by Haas et al. (1996). In the present example, a modified codon optimised HIV env sequence is used (SEQ I.D. No.3). The corresponding env expression plasmid is designated pSYNgp160 mn. The modified sequence contains extra motifs not used by Haas et al. The extra sequences were taken from the HIV env sequence of strain MN and codon optimised. Any similar modification of the nucleic acid sequence would function similarly as long as it used codons corresponding to abundant tRNAs (Zolotukhin et al., 1996) and lead to resistance to the ribozymes in the genome.

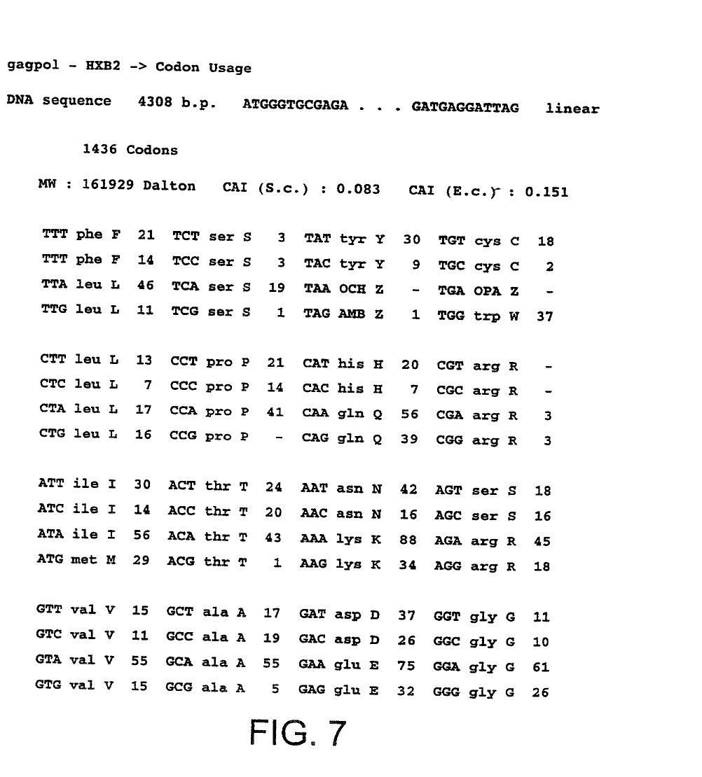

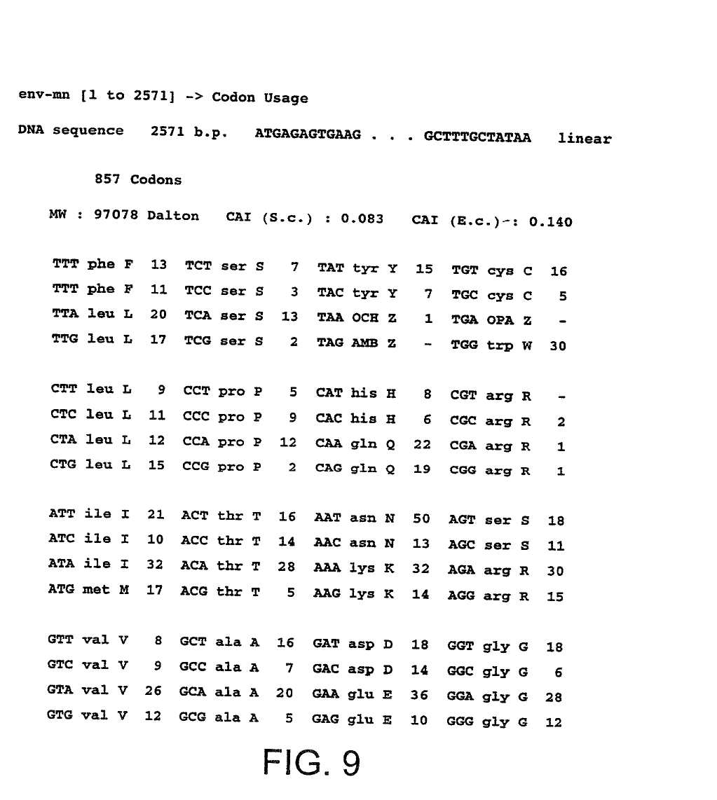

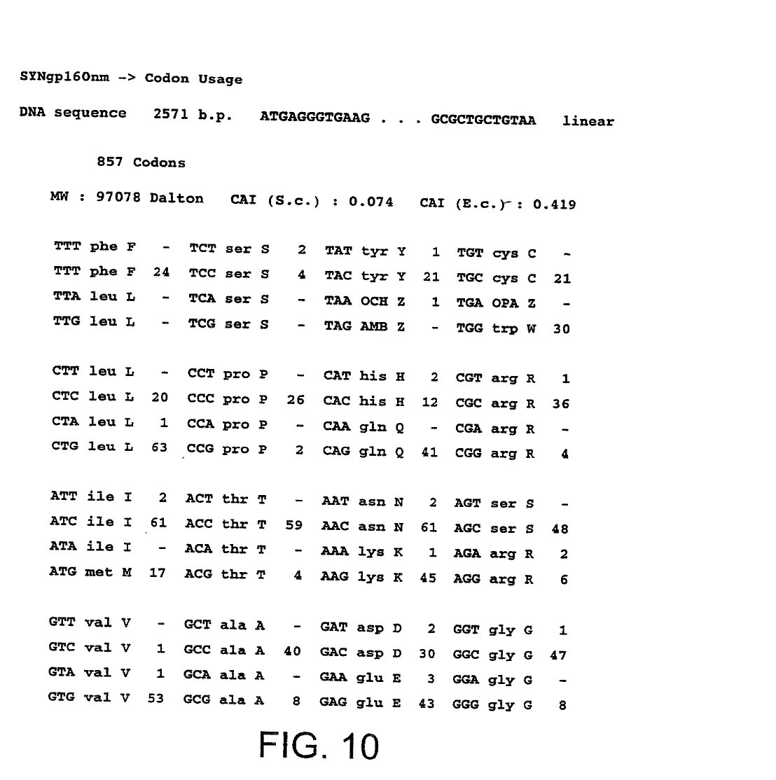

In one example of a gag, pol coding sequence with optimised codon usage, overlapping oligonucleotides are synthesised and then ligated together to produce the synthetic coding sequence. The sequence of a wild-type (Genbank accession no. K03455) and synthetic (gagpol-SYNgp) gagpol sequence is shown in SEQ I.D. Nos 1 and 2, respectively and their codon usage is shown in FIGS. 7 and 8, respectively. The sequence of a wild type env coding sequence (Genbank Accession No. M17449) is given in SEQ I.D. No 3, the sequence of a synthetic codon optimised sequence is given in SEQ. I.D. No. 4 and their codon usage tables are given in FIGS. 9 and 10, respectively. As with the env coding sequence any gag, pol sequence that achieves resistance to the ribozymes could be used. The synthetic sequence shown is designated gag, pol-SYNgp and has an EcoRI site at the 5′ end and a NotI site at the 3′ end. It is inserted into pClneo (Promega) to produce plasmid pSYNgp.

In a second form of the packaging system a synthetic gag, pol cassette is coexpressed with a non-HIV envelope coding sequence that produces a surface protein that pseudotypes HIV. This could be for example VSV-G (Ory et al., 1996: Zhu et al., 1990), amphotropic MLV env (Chesebro et al., 1990, Spector et al., 1990) or any other protein that would be incorporated into the HIV particle (Valsesia Wittmann et al., 1994). This includes molecules capable of targeting the vector to specific tissues. Coding sequences for non-HIV envelope proteins not cleaved by the ribozymes and so no sequence modification is required (although some sequence modification may be desirable for other reasons such as optimisation for codon usage in mammalian cells).

### Reference Example 3

### Vector Particle Production

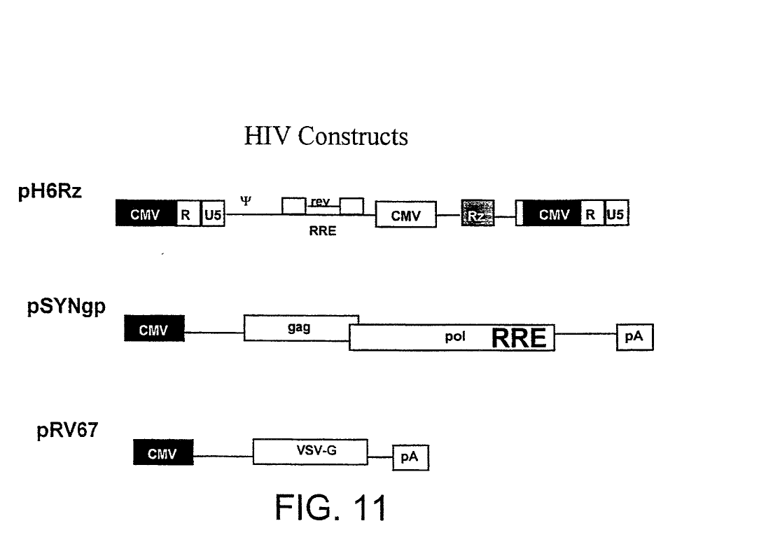

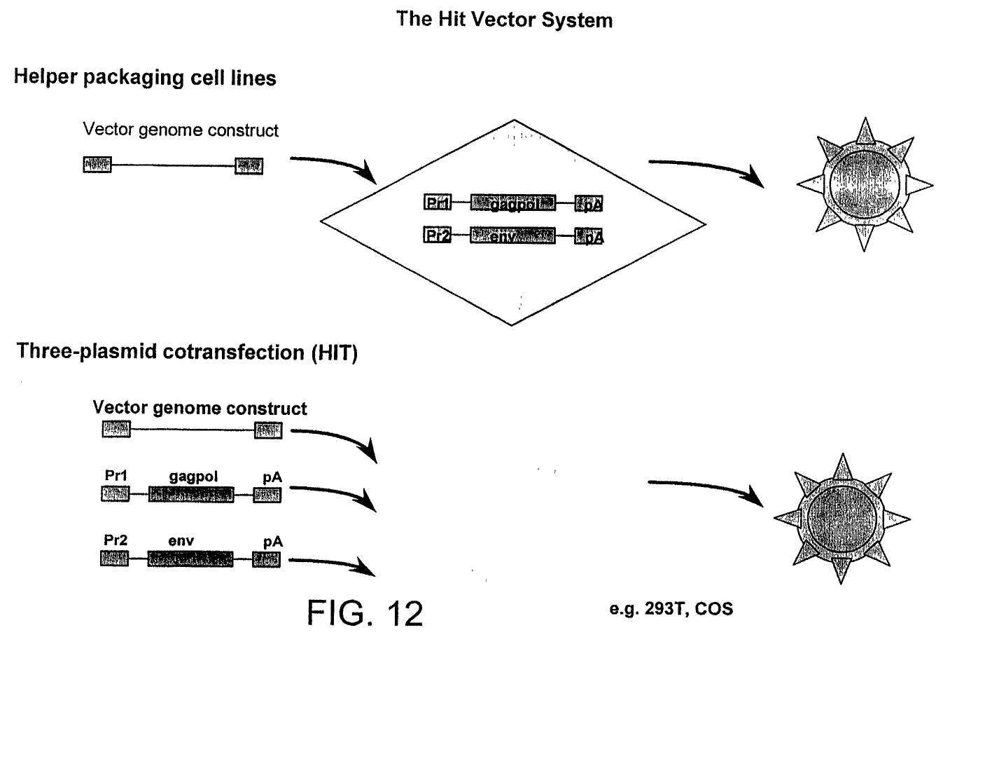

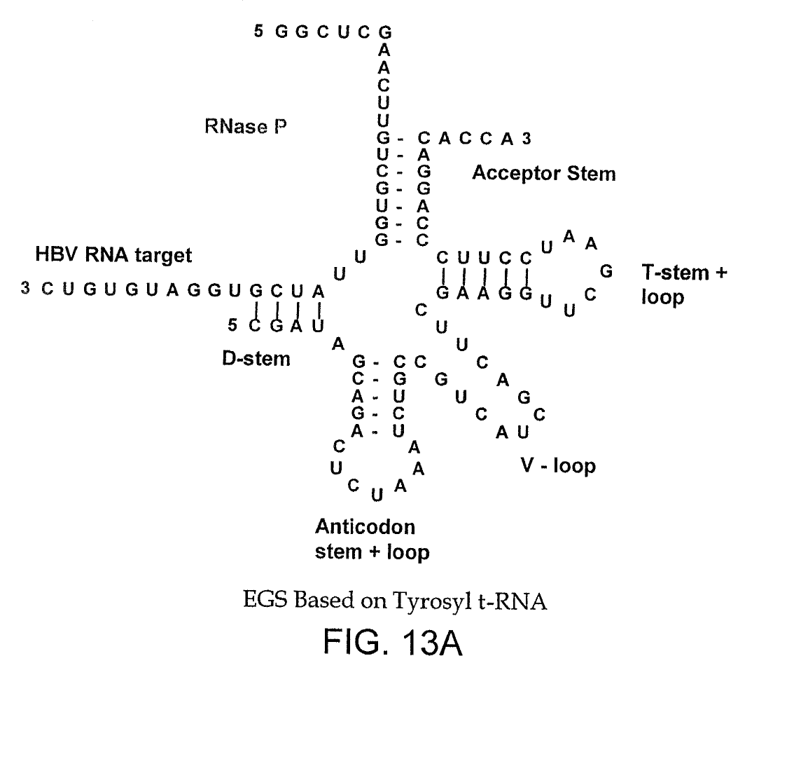

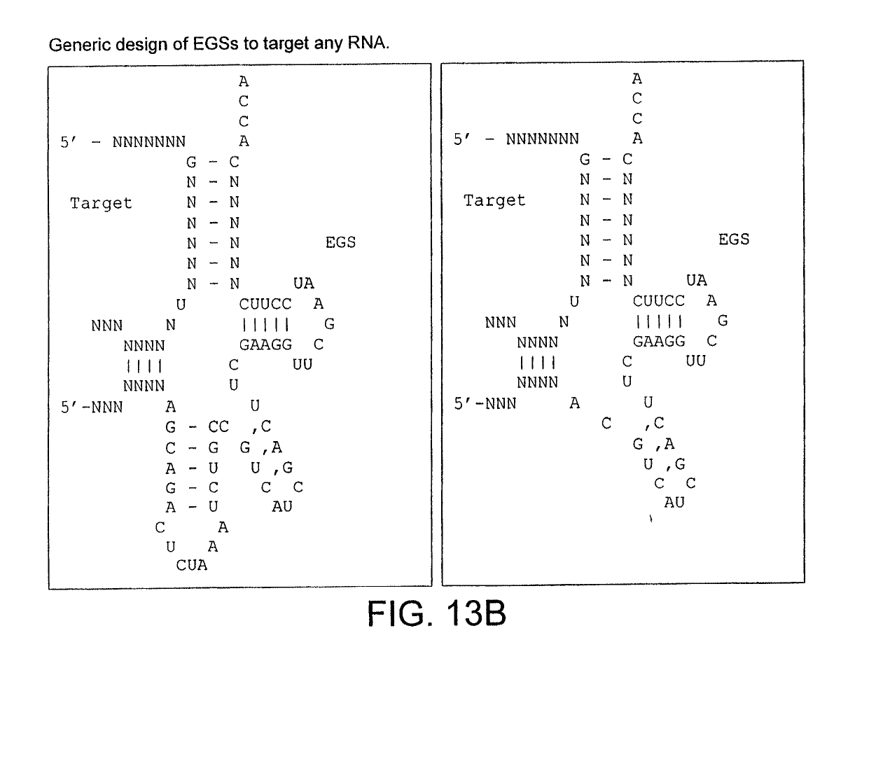

Vector particles can be produced either from a transient three-plasmid transfection system similar to that described by Soneoka et al. (1995) or from producer cell lines similar to those used for other retroviral vectors (Ory et al., 1996: Srinivasakumar et al., 1997; Yu et al., 1996). These principles are illustrated in FIGS. 11 and 12. For example, by using pH6Rz, pSYNgp and pRV67 (VSV-G expression plasmid) in a three plasmid transfection of 293T cells (FIG. 12), as described by Soneoka et al. (1995), vector particles designated H6Rz-VSV are produced. These transduce the H6Rz genome to CD4+ cells such as C1866 or Jurkat and produce the multitarget ribozymes. HIV replication in these cells is now severely restricted.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The invention will now be further described by the following numbered paragraphs:

1. A selection system suitable for use in vivo advantageously for selecting inhibitory RNA molecules, the system comprising:

(i) a plurality of first nucleotide sequences encoding a gene product capable of binding to and effecting the cleavage, directly or indirectly, of a nucleotide sequence, or a transcription product thereof;

wherein a region of the first nucleotide sequence required for binding to the nucleotide sequence is heterogeneous within the plurality of first nucleotide sequences; and

(ii) a second nucleotide sequence comprising

(a) a coding region encoding a detectable marker operably linked to sequences required for mRNA stability and/or translation; and

(b) a third nucleotide sequence positioned between the coding region and at least one of the sequences required for mRNA stability and/or translation;

wherein (a) and (b) are operably linked to a regulatory sequence capable of directing expression of (a) and (b) as a contiguous RNA molecule in a host cell; and advantageously wherein the first nucleotide sequence encoding a gene product is capable of binding to and effecting the cleavage, directly or indirectly, of the third nucleotide sequence or a transcription product thereof.

2. A vector system advantageously for selecting inhibitory RNA molecules comprising:

(i) a plurality of vectors, each vector independently comprising

a first nucleotide sequence encoding a gene product capable of binding to and effecting the cleavage, directly or indirectly, of a nucleotide sequence, or a transcription product thereof;

wherein a region of the first nucleotide sequence required for binding to the nucleotide sequence is heterogeneous within the plurality of vectors; and

(ii) a second nucleotide sequence comprising (a) a coding region encoding a detectable marker operably linked to sequences required for mRNA stability and/or translation; and (b) a third nucleotide sequence positioned between the coding region and at least one of the sequences required for mRNA stability and/or translation; wherein (a) and (b) are operably linked to a regulatory sequence capable of directing expression of (a) and (b) as a contiguous RNA molecule in a host cell, wherein the second nucleotide sequence is present in the plurality of vectors or as part of a separate vector; and advantageously wherein the first nucleotide sequence encoding a gene product is capable of binding to and effecting the cleavage, directly or indirectly, of the third nucleotide sequence or a transcription product thereof.

3. A system according to paragraph 1 or 2 wherein the gene product is selected from a ribozyme, an anti-sense ribonucleic acid and an external guide sequence.

4. A system according to paragraph 2 or 3 wherein advantageously the system is a system according to claim 2 and wherein the vector is a viral vector.

5. A system according to paragraph 4 wherein the viral vector is a retroviral vector.

6. A system according to any one of the preceding paragraphs wherein the detectable marker is a selectable marker.

7. A system according to paragraph 6 wherein the selectable marker is an enzyme capable of converting a prodrug into a cytotoxic compound.

8. A system according to paragraph 7 wherein the enzyme is thymidine kinase and the prodrug is gancyclovir.

9. A system according to any one of the preceding paragraphs wherein the third nucleotide sequence is present in the 3′ and/or 5′ untranslated region of the second nucleotide sequence.

10. A plurality of viral particles, each viral particle comprising a first nucleotide sequence according to paragraph 1 and/or a second nucleotide sequence according to paragraph 1.

11. A method for producing plurality of viral particles according to paragraph 10 which method comprises introducing into a producer cell (i) a plurality of first nucleotide sequences as defined in paragraph 1 and (ii) a second nucleotide sequence as defined in paragraph 1.

12. A plurality of viral particles produced by the method of paragraph 11.

13. A method for selecting from a plurality of first nucleotide sequences, a first nucleotide sequence encoding a gene product capable of binding to and effecting the cleavage, directly or indirectly, of a third nucleotide sequence or transcription product thereof; which method comprises:

(i) introducing the system according to claim 1 or claim 2 into a host cell; and

(ii) selecting the host cell if the detectable marker is not expressed in an active form in the host cell; and optionally,

(iii) isolating the first nucleotide sequence and determining its nucleotide sequence.

14. A method according to paragraph 13 wherein in step (ii), the detectable marker is a selectable marker and the host cell is contacted with a compound which is cytotoxic in the presence of the selectable marker.

15. A method according to paragraph 14 wherein the selectable marker is thymidine kinase and the compound is gancyclovir.

16. A method according to any one of paragraphs 13 to 1 5, which further comprises a selection step to remove any first nucleotide sequence(s) encoding a gene product capable of binding to and effecting the cleavage directly or indirectly of a coding region encoding the detectable marker.

17. A method according to paragraph 16, wherein each first nucleotide sequence in the plurality of first nucleotide sequences is located downstream of a fourth nucleotide sequence encoding an inactive variant of the detectable marker.

18. A method according to paragraph 16 or 17, wherein the plurality of first nucleotide sequences are present in a plurality of viral vectors, said viral vectors comprising a fourth nucleotide sequence encoding an inactive variant of the detectable marker, and as a preliminary step, the plurality of vectors are introduced into one or more producer cells and any resulting infectious viral particles used in step (i) to introduce the first nucleotides sequences into producer cells comprising a second nucleotide sequence.

19. A method for identifying an open site on a mRNA molecule, using a selection system according to any of paragraphs 1 to 9.

We claim:

1. A selection system suitable for use in vivo for selecting inhibitory RNA molecules, the system comprising:
(i) a plurality of first nucleotide sequences encoding a gene product capable of binding to and effecting the cleavage, directly or indirectly, of a nucleotide sequence, or a transcription product thereof;
wherein a region of the first nucleotide sequence required for binding to the nucleotide sequence is heterogeneous within the plurality of first nucleotide sequences; and
(ii) a second nucleotide sequence comprising:
(a) a coding region encoding a detectable marker operably linked to sequences required for mRNA stability and/or translation; and
(b) a third nucleotide sequence positioned between the coding region and at least one of the sequences required for mRNA stability and/or translation;
wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof.

2. A vector system for selecting inhibitory RNA molecules comprising:
(i) a plurality of vectors, each vector independently comprising
a first nucleotide sequence encoding a gene product capable of binding to and effecting the cleavage, directly or indirectly, of a nucleotide sequence, or a transcription product thereof;
wherein a region of the first nucleotide sequence required for binding to the nucleotide sequence is heterogeneous within the plurality of vectors; and
(ii) a second nucleotide sequence comprising:
(a) a coding region encoding a detectable marker operably linked to sequences required for mRNA stability and/or translation; and
(b) a third nucleotide sequence positioned between the coding region and at least one of the sequences required for mRNA stability and/or translation;
wherein (a) and (b) are operably linked to a regulatory sequence capable of directing expression of (a) and (b) as a contiguous RNA molecule in a host cell, wherein the second nucleotide sequence is present in the plurality of vectors or as part of a separate vector; and wherein the first nucleotide sequence encoding a gene product is capable of binding to and effecting the cleavage, directly or indirectly, of the third nucleotide sequence or a transcription product thereof.

3. A system according to claim 1 wherein the gene product is selected from a ribozyme, an anti-sense ribonucleic acid and an external guide sequence.

4. A system according to claim 2 wherein the system comprises:
(i) a plurality of vectors, each vector independently comprising
a first nucleotide sequence encoding a gene product capable of binding to and effecting the cleavage, directly or indirectly, of a nucleotide sequence, or a transcription product thereof;
wherein a region of the first nucleotide sequence required for binding to the nucleotide sequence is heterogeneous within the plurality of vectors; and
(ii) a second nucleotide sequence comprising:
(a) a coding region encoding a detectable marker operably linked to sequences required for mRNA stability and/or translation; and
(b) a third nucleotide sequence positioned between the coding region and at least one of the sequences required for mRNA stability and/or translation; wherein (a) and (b) are operably linked to a regulatory sequence capable of directing expression of (a) and (b) as a contiguous RNA molecule in a host cell, wherein the second nucleotide sequence is present in the plurality of vectors or as part of a separate vector; and wherein the first nucleotide sequence encoding a gene product is capable of binding to and effecting the cleavage, directly or indirectly, of the third nucleotide sequence or a transcription product thereof;
and wherein the vector is a viral vector.

5. A system according to claim 4 wherein the viral vector is a retroviral vector.

6. A system according to claim 1 wherein the detectable marker is a selectable marker.

7. A system according to claim 6 wherein the selectable marker is an enzyme capable of converting a prodrug into a cytotoxic compound.

8. A system according to claim 7 wherein the enzyme is thymidine kinase and the prodrug is gancyclovir.

9. A system according to claim 1 wherein the third nucleotide sequence is present in the 3′ and/or 5′ untranslated region of the second nucleotide sequence.

10. A plurality of viral particles, each viral particle comprising a first nucleotide sequence encoding a gene product capable of binding to and effecting the cleavage, directly or indirectly, of a nucleotide sequence, or a transcription product thereof; wherein a region of the first nucleotide sequence required for binding to the nucleotide sequence is heterogeneous within the plurality of first nucleotide sequences, and/or a second nucleotide sequence comprising
(a) a coding region encoding a detectable marker operably linked to sequences required for mRNA stability and/or translation; and
(b) a third nucleotide sequence positioned between the coding region and at least one of the sequences required for mRNA stability and/or translation;
wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof.

11. A method for producing plurality of viral particles, each viral particle comprising a first nucleotide sequence encoding a gene product capable of binding to and effecting the cleavage, directly or indirectly, of a nucleotide sequence, or a transcription product thereof; wherein a region of the first nucleotide sequence required for binding to the nucleotide sequence is heterogeneous within the plurality of first nucleotide sequences, and/or a second nucleotide sequence comprising
(a) a coding region encoding a detectable marker operably linked to sequences required for mRNA stability and/or translation; and
(b) a third nucleotide sequence positioned between the coding region and at least one of the sequences required for mRNA stability and/or translation;
wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof;
which method comprises introducing into a producer cell
(i) a plurality of first nucleotide sequences encoding a gene product capable of binding to and effecting the cleavage, directly or indirectly, of a nucleotide sequence, or a transcription product thereof; wherein a region of the first nucleotide sequence required for binding to the nucleotide sequence is heterogeneous within the plurality of first nucleotide sequences and
(ii) a second nucleotide sequence comprising:
(a) a coding region encoding a detectable marker operably linked to sequences required for mRNA stability and/or translation; and
(b) a third nucleotide sequence positioned between the coding region and at least one of the sequences required for mRNA stability and/or translation;
wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof.

12. A plurality of viral particles produced by the method comprising introducing into a producer cell
(i) a plurality of first nucleotide sequences encoding a gene product capable of binding to and effecting the cleavage, directly or indirectly, of a nucleotide sequence, or a transcription product thereof; wherein a region of the first nucleotide sequence required for binding to the nucleotide sequence is heterogeneous within the plurality of first nucleotide sequences and
(ii) a second nucleotide sequence comprising:
(a) a coding region encoding a detectable marker operably linked to sequences required for mRNA stability and/or translation; and
(b) a third nucleotide sequence positioned between the coding region and at least one of the sequences required for mRNA stability and/or translation;
wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof.

13. A method for selecting from a plurality of first nucleotide sequences, a first nucleotide sequence encoding a gene product capable of binding to and effecting the cleavage, directly or indirectly, of a third nucleotide sequence, or a transcription product thereof, which method comprises:
(i) introducing the system comprising:
(a) a plurality of first nucleotide sequences encoding a gene product capable of binding to and effecting the cleavage, directly or indirectly, of a nucleotide sequence, or a transcription product thereof;
wherein a region of the first nucleotide sequence required for binding to the nucleotide sequence is heterogeneous within the plurality of first nucleotide sequences; and
(b) a second nucleotide sequence comprising:
(i) a coding region encoding a detectable marker operably linked to sequences required for mRNA stability and/or translation; and
(ii) a third nucleotide sequence positioned between the coding region and at least one of the sequences required for mRNA stability and/or translation;
wherein (i) and (ii) are operably linked to a regulatory sequence capable of directing expression of (i) and (ii) as a contiguous RNA molecule in a host cell; and wherein the first nucleotide sequence encoding a gene product is capable of binding to and effecting the cleavage, directly or indirectly, of the third nucleotide sequence or a transcription product thereof;
into a host cell; and
(ii) selecting the host cell if the detectable marker is not expressed in an active form in the host cell; and optionally,
(iii) isolating the first nucleotide sequence and determining its nucleotide sequence.

14. A method according to claim 13 wherein in step (ii), the detectable marker is a selectable marker and the host cell is contacted with a compound which is cytotoxic in the presence of the selectable marker.

15. A method according to claim 14 wherein the selectable marker is thymidine kinase and the compound is gancyclovir.

16. A method according to claim 13, which further comprises a selection step to remove any first nucleotide sequence(s) encoding a gene product capable of binding to and effecting the cleavage directly or indirectly of a coding region encoding the detectable marker.

17. A method according to claim 16, wherein each first nucleotide sequence in the plurality of first nucleotide sequences is located downstream of a fourth nucleotide sequence encoding an inactive variant of the detectable marker.

18. A method according to claim 16, wherein the plurality of first nucleotide sequences are present in a plurality of viral vectors, said viral vectors comprising a fourth nucleotide sequence encoding an inactive variant of the detectable marker, and as a preliminary step, the plurality of vectors are introduced into one or more producer cells and any resulting infectious viral particles used in step (i) to introduce the first nucleotides sequences into producer cells comprising a second nucleotide sequence.

19. A method for identifying an open site on a mRNA molecule using a system comprising:
(i) a plurality of first nucleotide sequences encoding a gene product capable of binding to and effecting the cleavage, directly or indirectly, of a nucleotide sequence, or a transcription product thereof;
wherein a region of the first nucleotide sequence required for binding to the nucleotide sequence is heterogeneous within the plurality of first nucleotide sequences; and
(ii) a second nucleotide sequence comprising:
(a) a coding region encoding a detectable marker operably linked to sequences required for mRNA stability and/or translation; and
(b) a third nucleotide sequence positioned between the coding region and at least one of the sequences required for mRNA stability and/or translation; wherein (a) and (b) are operably linked to a regulatory sequence capable of directing expression of (a) and (b) as a contiguous RNA molecule in a host cell; and wherein the first nucleotide sequence encoding a gene product is capable of binding to and effecting the cleavage, directly or indirectly, of the third nucleotide sequence or a transcription product thereof.


SEQUENCE LISTING (26 sequences)

SEQ ID NO: 1 - DNA, 4307 bp, Human immunodeficiency virus type I
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 2 - DNA, 4307 bp, Artificial Sequence
  Codon optimised gagpol sequence
atgggcgcccgcgccagcgtgctgtcgggcggcgagctggaccgctgggagaagatccgcctgcgccccggcggcaaaaagaagtacaagctgaagcacatcgtgtgggccagccgcgaactggagcgcttcgccgtgaaccccgggctcctggagaccagcgaggggtgccgccagatcctcggccaactgcagcccagcctgcaaaccggcagcgaggagctgcgcagcctgtacaacaccgtggccacgctgtactgcgtccaccagcgcatcgaaatcaaggatacgaaagaggccctggataaaatcgaagaggaacagaataagagcaaaaagaaggcccaacaggccgccgcggacaccggacacagcaaccaggtcagccagaactaccccatcgtgcagaacatccaggggcagatggtgcaccaggccatctccccccgcacgctgaacgcctgggtgaaggtggtggaagagaaggcttttagcccggaggtgatacccatgttctcagccctgtcagagggagccaccccccaagatctgaacaccatgctcaacacagtggggggacaccaggccgccatgcagatgctgaaggagaccatcaatgaggaggctgccgaatgggatcgtgtgcatccggtgcacgcagggcccatcgcaccgggccagatgcgtgagccacggggctcagacatcgccggaacgactagtacccttcaggaacagatcggctggatgaccaacaacccacccatcccggtgggagaaatctacaaacgctggatcatcctgggcctgaacaagatcgtgcgcatgtatagccctaccagcatcctggacatccgccaaggcccgaaggaaccctttcgcgactacgtggaccggttctacaaaacgctccgcgccgagcaggctagccaggaggtgaagaactggatgaccgaaaccctgctggtccagaacgcgaacccggactgcaagacgatcctgaaggccctgggcccagcggctaccctagaggaaatgatgaccgcctgtcagggagtgggcggacccggccacaaggcacgcgtcctggctgaggccatgagccaggtgaccaactccgctaccatcatgatgcagcgcggcaactttcggaaccaacgcaagatcgtcaagtgcttcaactgtggcaaagaagggcacacagcccgcaactgcagggcccctaggaaaaagggctgctggaaatgcggcaaggaaggccaccagatgaaagactgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcagctcaaggaggctctcctggacaccggagcagacgacaccgtgctggaggagatgtcgttgccaggccgctggaagccgaagatgatcgggggaatcggcggtttcatcaaggtgcgccagtatgaccagatcctcatcgaaatctgcggccacaaggctatcggtaccgtgctggtgggccccacacccgtcaacatcatcggacgcaacctgttgacgcagatcggttgcacgctgaacttccccattagccctatcgagacggtaccggtgaagctgaagcccgggatggacggcccgaaggtcaagcaatggccattgacagaggagaagatcaaggcactggtggagatttgcacagagatggaaaaggaagggaaaatctccaagattgggcctgagaacccgtacaacacgccggtgttcgcaatcaagaagaaggactcgacgaaatggcgcaagctggtggacttccgcgagctgaacaagcgcacgcaagacttctgggaggttcagctgggcatcccgcaccccgcagggctgaagaagaagaaatccgtgaccgtactggatgtgggtgatgcctacttctccgttcccctggacgaagacttcaggaagtacactgccttcacaatcccttcgatcaacaacgagacaccggggattcgatatcagtacaacgtgctgccccagggctggaaaggctctcccgcaatcttccagagtagcatgaccaaaatcctggagcctttccgcaaacagaaccccgacatcgtcatctatcagtacatggatgacttgtacgtgggctctgatctagagatagggcagcaccgcaccaagatcgaggagctgcgccagcacctgttgaggtggggactgaccacacccgacaagaagcaccagaaggagcctcccttcctctggatgggttacgagctgcaccctgacaaatggaccgtgcagcctatcgtgctgccagagaaagacagctggactgtcaacgacatacagaagctggtggggaagttgaactgggccagtcagatttacccagggattaaggtgaggcagctgtgcaaactcctccgcggaaccaaggcactcacagaggtgatccccctaaccgaggaggccgagctcgaactggcagaaaaccgagagatcctaaaggagcccgtgcacggcgtgtactatgacccctccaaggacctgatcgccgagatccagaagcaggggcaaggccagtggacctatcagatttaccaggagcccttcaagaacctgaagaccggcaagtacgcccggatgaggggtgcccacactaacgacgtcaagcagctgaccgaggccgtgcagaagatcaccaccgaaagcatcgtgatctggggaaagactcctaagttcaagctgcccatccagaaggaaacctgggaaacctggtggacagagtattggcaggccacctggattcctgagtgggagttcgtcaacacccctcccctggtgaagctgtggtaccagctggagaaggagcccatagtgggcgccgaaaccttctacgtggatggggccgctaacagggagactaagctgggcaaagccggatacgtcactaaccggggcagacagaaggttgtcaccctcactgacaccaccaaccagaagactgagctgcaggccatttacctcgctttgcaggactcgggcctggaggtgaacatcgtgacagactctcagtatgccctgggcatcattcaagcccagccagaccagagtgagtccgagctggtcaatcagatcatcgagcagctgatcaagaaggaaaaggtctatctggcctgggtacccgcccacaaaggcattggcggcaatgagcaggtcgacaagctggtctcggctggcatcaggaaggtgctattcctggatggcatcgacaaggcccaggacgagcacgagaaataccacagcaactggcgggccatggctagcgacttcaacctgccccctgtggtggccaaagagatcgtggccagctgtgacaagtgtcagctcaagggcgaagccatgcatggccaggtggactgtagccccggcatctggcaactcgattgcacccatctggagggcaaggttatcctggtagccgtccatgtggccagtggctacatcgaggccgaggtcattcccgccgaaacagggcaggagacagcctacttcctcctgaagctggcaggccggtggccagtgaagaccatccatactgacaatggcagcaatttcaccagtgctacggttaaggccgcctgctggtgggcgggaatcaagcaggagttcgggatcccctacaatccccagagtcagggcgtcgtcgagtctatgaataaggagttaaagaagattatcggccaggtcagagatcaggctgagcatctcaagaccgcggtccaaatggcggtattcatccacaatttcaagcggaagggggggattggggggtacagtgcgggggagcggatcgtggacatcatcgcgaccgacatccagactaaggagctgcaaaagcagattaccaagattcagaatttccgggtctactacagggacagcagaaatcccctctggaaaggcccagcgaagctcctctggaagggtgagggggcagtagtgatccaggataatagcgacatcaaggtggtgcccagaagaaaggcgaagatcattagggattatggcaaacagatggcgggtgatgattgcgtggcgagcagacaggatgaggattag

SEQ ID NO: 3 - DNA, 2571 bp, Human Immunodeficiency virus type I
atgagagtgaaggggatcaggaggaattatcagcactggtggggatggggcacgatgctccttgggttattaatgatctgtagtgctacagaaaaattgtgggtcacagtctattatggggtacctgtgtggaaagaagcaaccaccactctattttgtgcatcagatgctaaagcatatgatacagaggtacataatgtttgggccacacaagcctgtgtacccacagaccccaacccacaagaagtagaattggtaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatactactaataccaataatagtactgctaataacaatagtaatagcgagggaacaataaagggaggagaaatgaaaaactgctctttcaatatcaccacaagcataagagataagatgcagaaagaatatgcacttctttataaacttgatatagtatcaatagataatgatagtaccagctataggttgataagttgtaatacctcagtcattacacaagcttgtccaaagatatcctttgagccaattcccatacactattgtgccccggctggttttgcgattctaaaatgtaacgataaaaagttcagtggaaaaggatcatgtaaaaatgtcagcacagtacaatgtacacatggaattaggccagtagtatcaactcaactgctgttaaatggcagtctagcagaagaagaggtagtaattagatctgagaatttcactgataatgctaaaaccatcatagtacatctgaatgaatctgtacaaattaattgtacaagacccaactacaataaaagaaaaaggatacatataggaccagggagagcattttatacaacaaaaaatataataggaactataagacaagcacattgtaacattagtagagcaaaatggaatgacactttaagacagatagttagcaaattaaaagaacaatttaagaataaaacaatagtctttaatcaatcctcaggaggggacccagaaattgtaatgcacagttttaattgtggaggggaatttttctactgtaatacatcaccactgtttaatagtacttggaatggtaataatacttggaataatactacagggtcaaataacaatatcacacttcaatgcaaaataaaacaaattataaacatgtggcaggaagtaggaaaagcaatgtatgcccctcccattgaaggacaaattagatgttcatcaaatattacagggctactattaacaagagatggtggtaaggacacggacacgaacgacaccgagatcttcagacctggaggaggagatatgagggacaattggagaagtgaattatataaatataaagtagtaacaattgaaccattaggagtagcacccaccaaggcaaagagaagagtggtgcagagagaaaaaagagcagcgataggagctctgttccttgggttcttaggagcagcaggaagcactatgggcgcagcgtcagtgacgctgacggtacaggccagactattattgtctggtatagtgcaacagcagaacaatttgctgagggccattgaggcgcaacagcatatgttgcaactcacagtctggggcatcaagcagctccaggcaagagtcctggctgtggaaagatacctaaaggatcaacagctcctggggttttggggttgctctggaaaactcatttgcaccactactgtgccttggaatgctagttggagtaataaatctctggatgatatttggaataacatgacctggatgcagtgggaaagagaaattgacaattacacaagcttaatatactcattactagaaaaatcgcaaacccaacaagaaaagaatgaacaagaattattggaattggataaatgggcaagtttgtggaattggtttgacataacaaattggctgtggtatataaaaatattcataatgatagtaggaggcttggtaggtttaagaatagtttttgctgtactttctatagtgaatagagttaggcagggatactcaccattgtcgttgcagacccgccccccagttccgaggggacccgacaggcccgaaggaatcgaagaagaaggtggagagagagacagagacacatccggtcgattagtgcatggattcttagcaattatctgggtcgacctgcggagcctgttcctcttcagctaccaccacagagacttactcttgattgcagcgaggattgtggaacttctgggacgcagggggtgggaagtcctcaaatattggtggaatctcctacagtattggagtcaggaactaaagagtagtgctgttagcttgcttaatgccacagctatagcagtagctgaggggacagatagggttatagaagtactgcaaagagctggtagagctattctccacatacctacaagaataagacagggcttggaaagggctttgctataa

SEQ ID NO: 4 - DNA, 2571 bp, Artificial Sequence
  Codon optimised env sequence
atgagggtgaaggggatccgccgcaactaccagcactggtggggctggggcacgatgctcctggggctgctgatgatctgcagcgccaccgagaagctgtgggtgaccgtgtactacggcgtgcccgtgtggaaggaggccaccaccaccctgttctgcgccagcgacgccaaggcgtacgacaccgaggtgcacaacgtgtgggccacccaggcgtgcgtgcccaccgaccccaacccccaggaggtggagctcgtgaacgtgaccgagaacttcaacatgtggaagaacaacatggtggagcagatgcatgaggacatcatcagcctgtgggaccagagcctgaagccctgcgtgaagctgacccccctgtgcgtgaccctgaactgcaccgacctgaggaacaccaccaacaccaacaacagcaccgccaacaacaacagcaacagcgagggcaccatcaagggcggcgagatgaagaactgcagcttcaacatcaccaccagcatccgcgacaagatgcagaaggagtacgccctgctgtacaagctggatatcgtgagcatcgacaacgacagcaccagctaccgcctgatctcctgcaacaccagcgtgatcacccaggcctgccccaagatcagcttcgagcccatccccatccactactgcgcccccgccggcttcgccatcctgaagtgcaacgacaagaagttcagcggcaagggcagctgcaagaacgtgagcaccgtgcagtgcacccacggcatccggccggtggtgagcacccagctcctgctgaacggcagcctggccgaggaggaggtggtgatccgcagcgagaacttcaccgacaacgccaagaccatcatcgtgcacctgaatgagagcgtgcagatcaactgcacgcgtcccaactacaacaagcgcaagcgcatccacatcggccccgggcgcgccttctacaccaccaagaacatcatcggcaccatccgccaggcccactgcaacatctctagagccaagtggaacgacaccctgcgccagatcgtgagcaagctgaaggagcagttcaagaacaagaccatcgtgttcaaccagagcagcggcggcgaccccgagatcgtgatgcacagcttcaactgcggcggcgaattcttctactgcaacaccagccccctgttcaacagcacctggaacggcaacaacacctggaacaacaccaccggcagcaacaacaatattaccctccagtgcaagatcaagcagatcatcaacatgtggcaggaggtgggcaaggccatgtacgccccccccatcgagggccagatccggtgcagcagcaacatcaccggtctgctgctgacccgcgacggcggcaaggacaccgacaccaacgacaccgaaatcttccgccccggcggcggcgacatgcgcgacaactggagatctgagctgtacaagtacaaggtggtgacgatcgagcccctgggcgtggcccccaccaaggccaagcgccgcgtggtgcagcgcgagaagcgggccgccatcggcgccctgttcctgggcttcctgggggcggcgggcagcaccatgggggccgccagcgtgaccctgaccgtgcaggcccgcctgctcctgagcggcatcgtgcagcagcagaacaacctcctccgcgccatcgaggcccagcagcatatgctccagctcaccgtgtggggcatcaagcagctccaggcccgcgtgctggccgtggagcgctacctgaaggaccagcagctcctgggcttctggggctgctccggcaagctgatctgcaccaccacggtaccctggaacgcctcctggagcaacaagagcctggacgacatctggaacaacatgacctggatgcagtgggagcgcgagatcgataactacaccagcctgatctacagcctgctggagaagagccagacccagcaggagaagaacgagcaggagctgctggagctggacaagtgggcgagcctgtggaactggttcgacatcaccaactggctgtggtacatcaaaatcttcatcatgattgtgggcggcctggtgggcctccgcatcgtgttcgccgtgctgagcatcgtgaaccgcgtgcgccagggctacagccccctgagcctccagacccggccccccgtgccgcgcgggcccgaccgccccgagggcatcgaggaggagggcggcgagcgcgaccgcgacaccagcggcaggctcgtgcacggcttcctggcgatcatctgggtcgacctccgcagcctgttcctgttcagctaccaccaccgcgacctgctgctgatcgccgcccgcatcgtggaactcctaggccgccgcggctgggaggtgctgaagtactggtggaacctcctccagtattggagccaggagctgaagtccagcgccgtgagcctgctgaacgccaccgccatcgccgtggccgagggcaccgaccgcgtgatcgaggtgctccagagggccgggagggcgatcctgcacatccccacccgcatccgccaggggctcgagagggcgctgctgtaa

SEQ ID NO: 5 - RNA, 22 nt, Artificial Sequence
  Catalytic domain of general ribozyme (Reference Example 1)
cugaugaggccgaaaggccgaa

SEQ ID NO: 6 - RNA, 22 nt, Human immunodeficiency virus type I
uaguaagaauguauagcccuac

SEQ ID NO: 7 - RNA, 22 nt, Human immunodeficiency virus type I
aacccagauuguaagacuauuu

SEQ ID NO: 8 - RNA, 22 nt, Human immunodeficiency virus type I
uguuucaauuguggcaaagaag

SEQ ID NO: 9 - RNA, 22 nt, Human immunodeficiency virus type I
aaaaagggcuguuggaaaugug

SEQ ID NO: 10 - RNA, 22 nt, Human immunodeficiency virus type I
acgaccccucgucacaauaaag

SEQ ID NO: 11 - RNA, 22 nt, Human immunodeficiency virus type I
ggaauuggagguuuuaucaaag

SEQ ID NO: 12 - RNA, 22 nt, Human immunodeficiency virus type I
auauuuuucaguucccuuagau

SEQ ID NO: 13 - RNA, 22 nt, Human immunodeficiency virus type I
uggaugauuuguauguaggauc

SEQ ID NO: 14 - RNA, 22 nt, Human immunodeficiency virus type I
cuuuggauggguuaugaacucc

SEQ ID NO: 15 - RNA, 22 nt, Human immunodeficiency virus type I
cagcuggacugucaaugacaua

SEQ ID NO: 16 - RNA, 22 nt, Human immunodeficiency virus type I
aacuuucuauguagauggggca

SEQ ID NO: 17 - RNA, 22 nt, Human immunodeficiency virus type I
aaggccgccuguuggugggcag

SEQ ID NO: 18 - RNA, 22 nt, Human immunodeficiency virus type I
uaagacagcaguacaaauggca

SEQ ID NO: 19 - DNA, 30 bp, Artificial Sequence
  5′ PCR primer (Reference Example 1)
cagctgctcgagcagctgaagcttgcatgc

SEQ ID NO: 20 - DNA, 34 bp, Artificial Sequence
  3′ PCR primer (Reference Example 1)
gtaagttatgtaacggacgatatcttgtcttctt

SEQ ID NO: 21 - DNA, 37 bp, Artificial Sequence
  3′ PCR primer (Reference Example 1)
cgcatagtcgacgggcccgccactgctagagattttc

SEQ ID NO: 22 - RNA, 34 nt, Hepatitis B virus
ggcucgaacuugucgugguuaucguggauguguc

SEQ ID NO: 23 - RNA, 63 nt, Artificial Sequence
  External Guide Sequence (Figure 13A)
cgauagcagacucuaaaucugccgucaucgacuucgaagguucgaauccuucccaggacacca

SEQ ID NO: 24 - RNA, 23 nt, Unknown
  Target RNA sequence (Figure 13B)
nnnnnnngnnnnnnunnnnnnnn

SEQ ID NO: 25 - RNA, 66 nt, Artificial Sequence
  External Guide Sequence (Figure 13B)
nnnnnnnagcagacucuaaaucugccgucaucgacuucgaagguucgaauccuucnnnnnncacca

SEQ ID NO: 26 - RNA, 49 nt, Artificial Sequence
  External Guide Sequence (Figure 13B)
nnnnnnnacgucaucgacuucgaagguucgaauccuucnnnnnncacca